(19) United States
(12) Patent Application Publication
(10) Pub. No.: US 2019/0307821 A1
(43) Pub. Date: Oct. 10, 2019

(54) METHODS AND COMPOSITIONS FOR CANCER THERAPY

(71) Applicant: Health Research, Inc., Buffalo, NY (US)

(72) Inventor: Danuta KOZBOR, Buffalo, NY (US)

(21) Appl. No.: 16/464,730

(22) PCT Filed: Nov. 29, 2017
(86) PCT No.: PCT/US17/63649

§ 371 Date: May 29, 2019

Related U.S. Application Data
(60) Provisional application No. 62/427,735, filed on Nov. 29, 2016.

(51) Int. Cl. A61K 35/768; A61K 31/704; A61P 35/00; C07K 14/52

(52) U.S. Cl. CPC A61K 35/768; A61K 31/704; A61P 35/00; C12N 2710/24143; C07K 2317/52; C12N 2710/24132; C07K 14/522

(57) ABSTRACT

Improved cancer therapies are provided and include methods for inhibiting growth of tumor cells in an individual by administering to the individual a polynucleotide encoding a protein that contains an immunoglobulin Fc and an antagonist peptide of a receptor expressed by tumor cells, and administering a chemotherapeutic agent to the individual, such that the growth of the tumor cells and/or metastasis of cancer cells is synergistically inhibited. Approaches are also provided for improving cancer therapies that include adoptive immunotherapies by using the polynucleotides to enhance tumor infiltration by immune cells.

### CROSS REFERENCE TO RELATED APPLICATIONS

This application claims priority to U.S. provisional patent application No. 62/427,735, filed Nov. 29, 2016, the disclosure of which is incorporated herein by reference.

### FIELD OF THE DISCLOSURE

The present disclosure relates generally to modulating immune responses and more specifically to enhancing cell-mediated immune response in an individual using Fc fusion proteins.

### BACKGROUND OF THE DISCLOSURE

The successful application of cancer vaccines to treat patients has remained elusive, and there is an ongoing and unmet need for improving the efficacy of compositions and methods that stimulate cell mediated and other immune response against antigens that are expressed by cancer cells. The present disclosure meets these and other related needs.

### SUMMARY

The present disclosure relates to improved cancer therapies. The disclosure includes methods for inhibiting growth of tumor cells and/or metastasis by enhancing the effects of chemotherapeutic agents and/or adoptive immunotherapies. In one approach a method of this disclosure involves administering to an individual in need of cancer therapy a polynucleotide encoding a protein that comprises an immunoglobulin Fc and an antagonist peptide of a receptor expressed by tumor cells. In embodiments, the antagonist peptide is a CXCR4 chemokine receptor antagonist that comprises or consists of the sequence KGVSLSYR (SEQ ID NO:1). In one embodiment, the peptide antagonist of CXCR4 comprises the sequence KGVSLSYR-K-RYSLSVGK (SEQ ID NO:2). In embodiments, the protein encoded by a polynucleotide used in methods of this disclosure encodes only one amino acid sequence of the antagonist peptide of the receptor expressed by the tumor cells. In embodiments, the polynucleotide encoding the protein is present in a recombinant oncolytic vaccinia virus. In certain embodiments, the viral vector encodes an Fc segment that is a human IgG1 Fc or human IgG3 Fc. In embodiments, the viral vector is administered systemically.

In certain approaches the disclosure provides for consecutively administering a polynucleotide and subsequently a chemotherapeutic agent to the individual. In certain approaches the polynucleotide sensitizes cancer cells and/or a tumor to the chemotherapeutic agent. In embodiments, this combination approach results in a synergizing inhibition of tumor growth and/or metastasis. In embodiments, the survival time of the individual is increased.

In certain approaches the disclosure provides for consecutively administering a polynucleotide described herein, and subsequently administering an adoptive immunotherapy to the individual. In embodiments this can enhance certain aspects of the immune response to the cancer, including but not necessarily limited to cell-mediated immune responses, including but not limited to enhancing tumor infiltration by immune cells. In embodiments, performing a method of this disclosure inhibits formation of tumor-immunosuppressive networks.

### DESCRIPTION OF THE FIGURES

FIG. 1. Phenotypic characterization of ID8-R and CAOV2-R ovarian tumor cells and their parental counterparts. Flow cytometry analysis of CD44 (a) and CXCR4 (b) expression in parental and drug-resistant variants was performed on single-cell suspensions with specific mAbs. Background staining was assessed using isotype control Abs. Data are from one representative experiment of three performed. (c) Susceptibility of ID8-R and CAOV2-R to vaccinia virus infection. The parental and drug-resistant tumor cells were cultured as a monolayer before infection with OVV-EGFP (MOI=1). The expression of EGFP in infected cells was examined under an immunofluorescence microscope 24 h later. Scale bars, 25 μm. One representative experiment of three performed is shown. (d) The number of EGFP-expressing cells in each culture was determined by examining single-cell suspensions 24 h after infection by flow cytometry analysis. Background staining depicts uninfected controls. One representative experiment of four independent experiments performed is shown. (e) Replication of OVV-EGFP in different cultures was determined by titrating viral particles released from the infected cells at different time points by plaque assays in CV-1 cell monolayers. Results are presented as the mean of plaque forming units (PFU)/million cells ±SD of three independent experiments performed in duplicate. *P<0.05, **P<0.01, and ***P<0.001. (f) Phosphorylation levels of Akt and ERK1/2 in tumor cells were determined by Western blotting with anti-phospho-Akt(5473-P), anti-phospho-Akt(T308-P) and anti-phospho-ERK1/2 (Thr202/Tyr204) Abs. Anti-total Akt and anti-total ERK1/2 Abs were used as internal controls and anti-GAPDH Ab was used as a loading control. Bands were developed with HRP-labeled secondary Abs followed by Clarity Western ECL detection system. Representative blot from one experiment out of three performed is shown.

FIG. 2. Cytotoxicity of vaccinia virus and DOX used alone or in combination. Cells plated in 96-well plates were treated with serial dilutions of OVV-EGFP (a) or DOX (b). Cell survival was determined after 72 h by 3-(4,5-Dimethylthiazol-2-yl)-2,5-diphenyltetrazolium bromide (MTT) assay and calculated using the following formula: % cell survival=(absorbance value of treated cells/absorbance value of untreated control cells)×100%. Each data point was generated from triplicate samples repeated twice. Results are presented as mean±SD. *P<0.05, **P<0.01, and ***P<0.001. The effect of combination treatments of vaccinia virus and DOX against parental (c) and resistant (d) ID8 and CAOV2 tumor cells. The virus (EC50) was added 12 h before treatment with serial dilutions of DOX (left panel), together with DOX (middle panel), or 12 h after DOX (right panel). Cell survival was determined after 72 h by MTT assay. Each data point was generated from triplicate samples repeated twice. Results are presented as mean±SD.

FIG. 3. Efficacy of oncolytic virotherapy and PLD treatment used alone and in combination against i.p. growth of ID8-R in syngeneic mice. (a) C57BL/6 mice (n=6-10) were injected i.p. with 2×10⁵ ID8-R cells. Oncolytic virotherapy with OVV-Fc (10⁸ PFU delivered i.p.) alone or in combination with PLD (10 mg/kg delivered i.v.) was initiated 10 days later. In parallel experiments, tumor-bearing mice were treated with PLD, or PLD was delivered to tumor bearing mice 8 days before or after virotherapy treatment. Control mice were treated with PBS. (b) Tumor progression was monitored by bioluminescence imaging using the Xenogen IVIS Imaging System. Data points represent mean±SD. (c) Survival was defined as the point at which mice were killed because of extensive tumor burden. Kaplan-Meier survival plots were prepared and significance was determined using the log-rank method. *P<0.05, **P<0.01, ***P<0.001.

FIG. 4. Virally-induced IFN-β expression augments DOX-induced apoptosis associated with increased surface exposure of surface CRT, phagocytosis of tumor cell debris by BM-derived DCs, and immunogenicity. Cell death in ID8-R tumor cells treated with OVV-Fc (MOI=1), DOX (1 μM) or OVV-Fc followed by DOX (12 h after infection) was determined by staining with Annexin V-FITC and LIVE/DEAD fixable violet to measure the induction of early apoptosis (Annexin V⁺/LIVE/DEAD fixable violet⁻) and late apoptosis/necrosis (Annexin V^{+/-}/LIVE/DEAD fixable violet⁺) by flow cytometry 24 h later. (a) One representative experiment of three independent experiments performed is shown. (b) Results are presented as the mean±SD of three independent experiments. *P<0.05, **P<0.01, ***P<0.001. (c) Culture supernatants were collected from OVV-Fc-infected ID8-R cells, filtered and treated with UV and psolaren (10 μg/ml). Culture supernatants collected from uninfected cells served as controls. The CVN media were added to uninfected ID8-R cultures alone or in combination with DOX and analyzed for induction of early apoptosis. The induction of early apoptosis in cultures treated with the CVN supernatant and DOX alone or in combination was inhibited by IFN-β blocking antibody (0.5 μg/ml). Data points represent mean±SD of three independent experiments. *P<0.05, **P<0.01. (d) Surface exposure of CRT in ID8-R (left panel) and CAOV2-R (right panel) cultures untreated or treated with OVV-Fc, DOX, or OVV-Fc and DOX combination was determined by flow cytometry after staining with an anti-CRT Ab or an isotype control 24 h after treatments. Results are presented as mean±SD of four independent experiments. **P<0.01. (e) Phagocytosis of cell-tracker-blue CMF₂HC-labeled tumor cells treated with OVV-Fc, DOX, or OVV-Fc and DOX combination by DCs was measured after 12 h by flow cytometry. All tumor cell cultures infected with vaccinia virus were treated with UV and psolaren to eliminate the virus before combining with DCs. Tumor cells receiving UV and psolaren treatment were included as additional controls. The percentages of CD11c-expressing DCs taking up tumor cells are indicated. One representative experiment of three independent experiments performed is shown. (f) In vivo anticancer vaccination. ID8-R cells cultured as described above were injected in one flank of five C57BL/6 mice per group. This was followed by injection of live tumor cells into the opposite flank 8 days later. Tumor growth was monitored by measuring s.c. tumor growth with microcaliper until control mice were euthanized due to extensive tumor burden. Results are presented as mean±SD of five independent experiments. *P<0.05, **P<0.01.

FIG. 5. Effect of the CXCR4-A-Fc fusion protein on ID8-R tumor growth. (a) Cell death in ID8-R tumor cells treated with soluble CXCR4-A-Fc fusion protein (100 μg/ml) for 24 h was determined by staining with Annexin V-FITC and LIVE/DEAD fixable violet. Tumor cells treated with soluble Fc fragment of mouse IgG2a serve as controls. One representative experiment of three independent experiments performed is shown. (b) C57BL/6 mice (n=8-10) were injected i.p. with 2×10⁵ ID8-R cells. Oncolytic virotherapy with OVV-CXCR4-A-Fc or OVV-Fc (10⁸ PFU delivered i.p.) was initiated 10 days later. In parallel experiments, tumor-bearing mice were treated with PLD (10 mg/kg) delivered i.v. or PLD was delivered to virally-treated mice 8 days after virus injection. Control mice were treated with PBS. Tumor progression was monitored by bioluminescence imaging using the Xenogen IVIS Imaging System. Data points represent mean±SD. (c) Kaplan-Meier survival plots were prepared and significance was determined using the log-rank method. *P<0.05, **P<0.01, ***P<0.001. (d) Metastatic dissemination in the omentum, diaphragm, mesentery and peritoneal wall was assessed by identifying metastatic colonies (>5 mm) in individual mice at the time of development of bloody ascites in control mice. Representative images of metastasis within the peritoneal cavity of one mouse from each group are shown.

FIG. 6. Evaluation of immune infiltrates in ascites-derived tumors or peritoneal washes by combination treatments. Frequencies of G-MDSCs (CD11b⁺Ly6C^{low}Ly6G⁺) (a), Tregs (CD4⁺CD25⁺Foxp3⁺) (b) in ascites-derived tumors of control and treated mice were analyzed by flow cytometry as described in the Materials and Methods section. Results are presented as mean±SD of five mice per group. *P<0.05, **P<0.01. (c) The percent of CD11c⁺CD86⁺DCs and CD11b⁺F4/80⁺monocytes/macrophages in ascites derived tumors of the same groups of mice as above were analyzed by flow cytometry. The expression of IL-12 and IL-10 in CD11b⁺F4/80⁺ cells was determined by intracellular staining. Data points represent mean±SD. Statistically significant changes are bolded. (d) The ratios of IFN-γ-expressing CD8⁺ T cells/Tregs in ascites-derived tumors were determined by intracellular staining with mAbs against IFN-γ-PE and CD8-PECy5 together with mAbs against Tregs (CD4⁺CD25⁺Foxp3⁺) and flow cytometry of five mice per group. Data points represent mean±SD. *P<0.05, ***P<0.001. (e) The percent of WT1126-134 tetramer-specific CD8⁺ T cells was determined by staining with anti-CD8-PECy5 mAb and PE-labeled H-2D^{b}-restricted WT1126-134 tetramer. Background staining was assessed using isotype control antibodies. One representative experiment of five mice per group performed is shown. (f) Results are presented as mean±SD of five mice per group. *P<0.05, ***P<0.001.

FIG. 7. Graphical summary of improved long-term tumor-free survival by treatment of drug-resistant ovarian tumors in vivo by oncolytic virotherapy followed by PLD. (a) Intraperitoneal injection with OVV-CXCR4-A-Fc stimulates anticancer immunity through CXCR4-A-Fc-mediated inhibition of immunosuppressive cell recruitment, releases of PAMPs and DAMPs and immune cell infiltration, while also causing direct cellular cytotoxicity. (b) Treatment with PLD inhibits tumor growth through induction of immunogenic cell death, weakly effective in the drug-resistant mutants. (c) The synergistic interaction of OVV with PLD augments tumor cell death and inflammation, thus potentially increasing immunogenicity of endogenous tumor-associated antigens (TAAs). Low responses (+), medium responses (++), high responses (+++).

FIG. 8. Growth characteristics of drug-resistant tumor cells. Differences in growth rates between parental (P) and the drug-resistant (R) tumor variants of ID8 (a) and CAOV2 (b) cells were determined by a trypan blue exclusion test for cell viability. *P<0.05.

FIG. 9. Susceptibility of parental and resistant ID8 and CAOV2 tumor cells to PTX, CBDCA and DOX. (a) Characterization of side population (SP) in the parental (P) and drug-resistant (R) ID8 (left panel) and CAOV2 (right panel) tumor cells by Hoechst 33342 dye staining. One representative experiment of two independent experiments performed in duplicates is shown. Growth of the parental and drug-resistant tumor variants of ID8 (b) and CAOV2 (c) cells in the presence of PTX (59 nM) and CBDCA (2.6 μM) (left panel) or DOX (3 μM) (right panel) was determined by a trypan blue exclusion test for cell viability. Bars represent the mean±SD of two independent experiments using triplicate samples.

FIG. 10. Vaccinia virus replication in infected cultures treated with DOX. (a) ID8-Rand CAOV2-R tumor cells were infected with OVV-EGFP (MOI=3) and treated 12 h later with DOX (3 μM). Replication of OVV-EGFP was determined by titrating viral particles released from the infected cells after 24 h by plaque assays in CV-1 cell monolayers. Results were presented as mean±SD of two independent experiments performed in duplicates. *P<0.05 and ***P<0.001. (b) The uninfected and virally-infected cultures were lysed and analyzed by Western blotting with vaccinia virus-specific mouse antiserum (dilution 1:2,000). Normal mouse serum at 1:2,000 dilution was used as specificity control and anti-GAPDH Ab was used as a loading control. Bands were developed with HRP-labeled secondary Abs followed by Clarity Western ECL detection system. One representative blot of two performed is shown.

FIG. 11. Cytotoxicity of OVV-EGFP and OVV-Fc against ID8-R and CAOV2-R cells. Cells plated in 96-well plates were treated with serial dilutions of OVV-EGFP or OVV-Fc. Cell survival was determined after 72 h by MTT assay and calculated using the following formula: % cell survival=(absorbance value of treated cells/absorbance value of untreated control cells)×100%. Each data point was generated from triplicate samples. Results were presented as mean±SD.

FIG. 12. Effect PLD treatment on the efficacy of OVV-Fc when used simultaneously or 12 h after virotherapy treatment against i.p. growth of ID8-R in syngeneic mice. (a) C57BL/6 mice (n=4-5) were injected i.p. with 2×105 ID8-R cells. Oncolytic virotherapy with OVV-Fc (108 PFU delivered i.p.) alone or in combination with PLD (10 mg/kg delivered i.v.) was initiated 10 days later. PLD was delivered simultaneously with OVV-Fc or 12 h later. (b) Tumor progression was monitored by bioluminescence imaging using the Xenogen IVIS Imaging System. Data points represent mean±SD.

FIG. 13. Vaccinia infection sensitizes CAOV2-R tumor cells to DOX-induced apoptosis. (a) Cell death in CAOV2-R tumor cells treated with OVV-Fc (MOI=1), DOX (1 μM) or OVV-Fc followed by DOX (12 h after infection) was determined by staining with Annexin V-FITC and LIVE/DEAD fixable violet to measure the induction of early apoptosis (Annexin V+/LIVE/DEAD fixable violet-) and late apoptosis/necrosis (Annexin V+/−/LIVE/DEAD fixable violet+) by flow cytometry 24 h later. (a) One representative experiment of three independent experiments performed is shown. (b) Results are presented as the mean±SD of three experiments. *P<0.05, **P<0.01, ***P<0.001.

FIG. 14. Induction of cell death by soluble CXCR4-A-Fc fusion proteins in CAOV2-R cells. Cells were treated with 100 μg/ml of a soluble Fc fragment of murine IgG2a (a), CXCR4-A-Fc fusion protein with the Fc fragment corresponding to murine IgG2a (b) and CXCR4-A-hFc fusion protein with the Fc fragment corresponding human IgG1 (c). After 24 h of incubation, the induction of apoptosis/necrosis was determined by staining with Annexin V-FITC and LIVE/DEAD fixable violet. One representative experiment of three experiments performed is shown.

FIG. 15. Inhibition of ID8-R tumor growth by adoptive transfer of splenocytes from tumor-free mice with detectable WT1-specific T cell responses to ID8-R-bearing mice after combining them with LPS-matured WT1126-134 peptide-pulsed BM-derived DCs. For the adoptive transfer, three C57BL/6 mice were injected s.c. with 105 ID8-R cells and treated 10 days later by i.v. injection of 2×107 splenocytes from OVV-CXCR4-A-Fc- and PLD-treated, tumor-free mice after stimulation with WT1126-134 peptide-coated DCs. Tumor growth was monitored by measuring s.c. tumors once to thrice a week with a microcaliper. Results are presented as the mean±SD of three independent experiments. **P<0.01.

FIG. 16. Effect of oncolytic virotherapy and PLD on orthotopic CAOV2-R tumor growth. (a) SCIDmice (n=6) were injected i.p. with 2×106 CAOV2-R cells. Oncolytic virotherapy with OVV-CXCR4-A-Fc, OVV-Fc (2.5×107 PFU delivered i.p.) or PLD (5 mg/kg) delivered i.v. was initiated 10 days later. In parallel experiments, PLD was delivered to virally-treated mice 8 days after virus injection. Control mice were treated with PBS. Tumor progression was monitored by bioluminescence imaging. (b) Kaplan-Meier survival plots were prepared and significance was determined using the log-rank method. *P<0.05, **P<0.01, ***P<0.001.

### DESCRIPTION OF THE DISCLOSURE

Unless defined otherwise herein, all technical and scientific terms used herein have the same meaning as commonly understood by one of ordinary skill in the art to which this disclosure pertains.

Unless specified to the contrary, it is intended that every maximum numerical limitation given throughout this description includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The disclosure includes all effects on cancer cells and tumors, and all combinations of them, that are described herein. The disclosure includes demonstrating an advantage of embodiments of the disclosure by comparison of a result obtained by practicing a method of this disclosure to any suitable control. Controls used with the present disclosure can comprise any suitable reference, including but not limited to a standardized value, an area under a curve, a value taken before cancer treatment, or a value obtained from a different combination of therapies. In certain embodiments the value represents at least two measurements, wherein one measurement is taken before therapy, and a second measurement is taken after during or after the conclusion of therapy.

This application incorporates by reference the disclosures of U.S. Pat. No. 9,296,803, PCT/US2011/028070, and improves upon those disclosures by providing for enhanced cancer treatment by combining oncolytic viral vectors with other cancer therapies, including but not necessarily limited to chemotherapies and adoptive immunotherapy approaches. Thus, in general, the present disclosure provides improved approaches for inhibiting growth of cancer cells in an individual in need thereof. In a non-limiting embodiment the present disclosure relates to inhibition of the growth of cancer. In embodiments, the disclosure is pertinent to inhibition of metastatic growth of ovarian tumor variants that are resistant to chemotherapeutic agents, such as paclitaxel and/or carboplatin, or doxorubicin, or other chemotherapeutic agents, including but not necessarily limited to other platinum-based agents. Synergistic effects are demonstrated.

In embodiments, the disclosure relates to improvement of cell-mediated immune therapy, including but not necessarily limited to adoptive immunotherapies. In embodiments the disclosure results in inhibition of the formation of an intratumor suppressive network. In embodiments, the disclosure facilitates improved dendritic cell participation in cell-mediated anti-cancer responses. In embodiments, the disclosure facilitates and/or enhances immune cell infiltration into tumors, including but not necessarily CD8+ T cells. In embodiments, the disclosure enhances cancer cell death.

In an embodiment, inhibition of growth of cancer cells and/or metastasis is achieved by a recombinant viral vector, such as oncolytic vaccinia virus, expressing a CXCR4 antagonist to target the CXCL12 chemokine/CXCR4 receptor signaling axis. Non-limiting embodiments are demonstrated using the viral constructs alone and in combination with the representative chemotherapeutic agent doxorubicin. The resistant variants exhibited augmented expression of the hyaluronan receptor CD44 and CXCR4 along with elevated Akt and ERK1/2 activation and displayed an increased susceptibility to viral infection compared with the parental counterparts. The infected cultures were more sensitive to doxorubicin-mediated killing both in vitro and in tumor-challenged mice. The combination treatment increased apoptosis and phagocytosis of tumor material by dendritic cells associated with induction of antitumor immunity. Targeting syngeneic tumors with this regimen increased intratumoral infiltration of antitumor CD8+ T cells. Thus, approaches of this disclosure are pertinent to enhancing cell-mediated immunity. The results were further enhanced by reducing the immunosuppressive network by the virally-delivered CXCR4 antagonist, which augmented antitumor immune responses and led to tumor-free survival. The results described herein thus define novel strategies for treatment of drug-resistant ovarian cancer that increase immunogenic cell death and reverse the immunosuppressive tumor microenvironment, culminating in antitumor immune responses that control metastatic tumor growth. All of these embodiments are encompassed in this disclosure.

In one embodiment, the method comprises administering to the individual a composition comprising a polynucleotide encoding an immunoglobulin (Ig) Fc and an antigen expressed by the cells or a mimotope of the antigen. In another embodiment, the method comprises administering to the individual a composition comprising a polynucleotide encoding an immunoglobulin Fc and an antagonist peptide of a CXCR4 chemokine receptor expressed by the cells. In various embodiments, the disclosure inhibits the growth of cancer cells, which can be but are not necessarily limited to tumor cells.

The polynucleotide which encodes the Ig Fc is in one embodiment a recombinant oncolytic vaccinia virus.

The fusion proteins encoded by the polynucleotides can comprise a human IgG1 Fc or human IgG3 Fc, and can further comprise T helper epitopes. The disclosure also provides compositions comprising polynucleotides encoding the proteins, and/or the encoded proteins. The proteins comprise an immunoglobulin Fc and an antigen or a peptide mimic of the antigen, or an

The present disclosure takes advantage of our discovery that a fusion protein comprising an Fc region of an antibody and a peptide antagonist of a receptor expressed on cancer cells can be used to overcome resistance to certain chemotherapeutic agents, and can enhance certain cell-mediated anti-tumor effects, as more fully described below and in the figures that accompany this disclosure. In certain embodiments the disclosure thus comprises fusion proteins and methods of using such fusion proteins, as well as compositions comprising such fusion proteins, compositions comprising polynucleotides encoding such fusion proteins, and methods of using such compositions for prophylaxis and/or therapy of disease, and to enhance chemotherapeutic and adoptive immunotherapy approaches to treating cancer. The method in general comprises administering a composition of the disclosure to an individual such that the growth of cells that express the receptor to which the peptide antagonist binds is inhibited, and/or that metastasis of cancer cells that express the receptor is inhibited, and/or that cancer cells that express the receptor are sensitized to one or more chemotherapeutic drugs, and/or become more susceptible to cell-mediated immunity. Thus, in embodiments the disclosure comprises enhancing the effect of a chemotherapeutic agent. Such agents include but are not limited to doxorubicin (DOX), including various DOX-containing formulations. In embodiments, the disclosure comprises enhancing the effect of an adoptive immunotherapy. Those skilled in the art will recognize that adoptive immunotherapy generally involves providing a patient with T cells, which may be unmodified or modified T cells. Adoptive immunotherapy is also described in the literature, such as in Restifo, et al. Adoptive immunotherapy for cancer: harnessing the T cell response Nature Reviews Immunology 12, 269-281 (2012), the disclosure of which is incorporated herein by reference.

In certain aspects the disclosure comprises selecting an individual who has a cancer that is partially or fully resistant to a chemotherapeutic agent and administering a composition of this disclosure and the chemotherapeutic agent to the individual. The composition of the disclosure and the chemotherapeutic agent can be administered sequentially or concurrently provided that the effect of the chemotherapeutic agent is enhanced. In an embodiment, a composition of this disclosure is administered prior to a chemotherapeutic agent. In embodiments, administration of a composition of this disclosure results in any one or any combination of: a synergistic increase involving direct oncolysis of resistant variants, a decrease in intratumoral recruitment of immunosuppressive elements, and/or stimulation of antitumor immunity, including but not limited to antitumor immunity that leads to curative inhibition of tumor growth.

Certain implementations of this disclosure employs an anticancer agent (CTCE-9908) that is a CXCR4 chemokine receptor antagonist. This peptide antagonist of CXCR4 comprises the amino acid sequence: KGVSLSYR-K-RYSLSVGK (SEQ ID NO:2). Thus, in one embodiment, the peptide antagonist of CXCR4 comprises the sequence KGVSLSYR (SEQ ID NO:1). In embodiments, there is only a single copy of the peptide antagonist present in a composition of the disclosure, and/or only a single copy of the peptide antagonist encoded by a vector used in embodiments of this disclosure.

CTCE-9908 blocks the interaction of the CXCR4 receptor with CXCL12, which is critical in the infiltration of organ tissue by metastatic cells, thereby reducing tumor metastasis. CXCR4 receptors are expressed on many tumor cell types. By performing the method of the disclosure, we take advantage of the Fc fragments naturally present disulfide bonds to preserve the dimeric structure of the CTCE-9908 peptide. The CTCE-9908 peptide can be expressed in the context of the activating murine and human Fcγ fragments (IgG2a and IgG1, respectively) in DNA vectors and can augment therapeutic efficacy of the peptide by mobilizing Antibody-Dependent Cell-Mediated Cytotoxicity (ADCC) tumor-killing mechanism. Accordingly, certain embodiments of the disclosure provides a composition comprising a fusion construct, wherein the fusion construct comprises an Fc region of a murine IgG2a or human IgG1 or a fragment of such Fc regions. In various embodiments, the Fc region is an Fc region or fragments thereof that is from an IgA, IgG, or IgE antibody, although Fc regions from other antibody types, or synthetic/artificial Fc regions can also be used. The Fc region can comprise or consist of an amino acid sequence that is identical to an Fc region produced by a mammal, such as a human. In various embodiments, the Fc region may have between 80% to 100% (including all integers there between) amino acid sequence similarity to an Fc region produced by a mouse and/or a human. The Fc region may be an intact Fc region, meaning an entire Fc region, or may be a fragment of the Fc region. Fragments of the Fc region preferably comprise amino acid sequences that specifically bind to Fcγ receptors. Those skilled in the art will recognize that the “Fc region” of an antibody means the “Fragment, crystallizable” region of the antibody, which comprises two heavy chains that contribute two or three constant domains (CD) depending on the class of the antibody. Nucleotide sequences encoding Fc regions, as well as the amino acid sequences of Fc regions for mouse and human immunoglobulins are well known in the art. In one embodiment, a suitable human Ig gamma-1 C region, Homo sapiens, for use as the Ig region in the instant disclosure has the sequence

(SEQ ID NO: 3)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLEPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYT.

In another embodiment, a suitable human Ig gamma-3 chain C region has the

(SEQ ID NO: 4)
ASTKGPSVFPLAPCSRSTSGGTAALGCLVKDYFPEPVTVSWNSGALTSGV
HTFPAVLQSSGLYSLSSVVTVPSSSLGTQTYTCNVNHKPSNTKVDKRVEL
KTPLGDTTHTCPRCPEPKSCDTPPPCPRCPEPKSCDTPPPCPRCPEPKSC
DTPPPCPRCPAPELLGGPSVFLFPPKPKDTLMISRTPEVTCVVVDVSHED
PEVQFKWYVDGVEVHNAKTKPREEQYNSTFRVVSVLTVLHQDWLNGKEYK
CKVSNKALPAPIEKTISKTKGQPREPQVYTLPPSREEMTKNQVSLTCLVK
GFYPSDIAVEWESSGQPENNYNTTPPMLDSDGSFFLYSKLTVDKSRWQQG
NIFSCSVMHEALHNRFTQKSLSLSPGK.

Because individual antibody isotypes possess different affinities for Fcγ receptors (with activating Fcγ receptors having higher affinities for murine IgG2a and IgG2b isotypes or human IgG1 and IgG3 isotypes), differences in the ratios of activating-to-inhibitory receptor binding by the presented antigenic complex may predict the ability of DCs to induce immune responses. Harnessing this pathway may allow the recruitment of adaptive immunity and immunologic memory by antibody therapy or cancer vaccines.

In one embodiment, the Fc portion of the fusion proteins comprises only antibody heavy chain(s).

Those skilled in the art will recognize that for demonstration of the disclosure using murine animal models, the Fc portion of the fusion protein is preferably an IgG2a or IgG2b Fc murine Ig portion, while for therapy and/or prophylaxis of disease in humans, the Fc portion is preferably an IgG1 or an IgG3 Fc portion.

In certain embodiments, the Fc portion of the fusion proteins provided herein do not include antigen recognition portions (i.e., the antibody portion of the fusion proteins do not contain antibody variable regions). Thus, the fusion proteins are distinct from antibodies that do contain antigen binding portions, and which may also include cross-linked or otherwise connected mimotopes, antigens, or peptide receptor ligands.

DNA constructs encoding the Fc-fusion proteins can be made using any conventional techniques well known to those skilled in the art. For example, the Fc-fusion encoding constructs can be made using commercially available reagents. In embodiments the Fc region comprises the CH2 and CH3 domains of the IgG heavy chain and the hinge region. The hinge acts as a flexible spacer between the two parts of the Fc-fusion protein, which permits each part of the fusion protein to function independently. In general, any Fc region (and accordingly any polynucleotides encoding such Fc region) that activates Fey receptors can be used in performance of the disclosure.

The DNA constructs encoding the fusion proteins can be expressed to produce the fusion proteins for isolation and/or purification, or for therapeutic purposes, using any suitable protein expression system. Various tags or other moieties can be added to the fusion proteins so that they can be readily purified using, for example, various affinity chromatography methods.

For therapeutic purposes, compositions such as pharmaceutical preparations comprising the fusion proteins, and/or comprising polynucleotides encoding the fusion proteins, can be prepared. Compositions for use in therapeutic purposes may be prepared by mixing the Fc-fusion proteins and/or polynucleotides encoding them with any suitable pharmaceutically acceptable carriers, excipients and/or stabilizers. Some examples of compositions suitable for mixing with the agent can be found in: Remington: The Science and Practice of Pharmacy (2005) 21st Edition, Philadelphia, Pa. Lippincott Williams & Wilkins. The compositions may further comprise any suitable adjuvant.

If the therapeutic agent used in the method of the disclosure is a polynucleotide, it can be administered to the individual as a naked polynucleotide, in combination with a delivery reagent, or as a recombinant plasmid or viral vector which comprises and/or expresses the polynucleotide agent. Suitable delivery reagents for administration include the Minis Transit TKO lipophilic reagent; lipofectin; lipofectamine; cellfectin; or polycations (e.g., polylysine), or liposomes. In one embodiment, the Fc-antagonist fusion is encoded by a recombinant oncolytic recombinant vaccinia virus (rOVV).

Those skilled in the art will recognize how to formulate dosing regimens for performing the method of the disclosure, taking into account such factors as the size and age of the individual to be treated, and the type and stage of a disease with which the individual may be suspected of having or may have been diagnosed with. The disclosure may be used to elicit an enhanced immune response that is prophylactic or therapeutic. The individual to whom the composition is administered can be an individual in need of the treatment, and/or an individual who has been diagnosed with, is suspected of having, or is at risk for developing a disease or other disorder that is associated with expression of the antigen.

The amount of Fc-antagonist fusion protein, or expression vector encoding the Fc-antagonist protein, or cells, such as antigen presenting cells comprising the Fc-antagonist fusion protein or an expression vector or other expression cassette encoding the Fc-antagonist fusion, to be included in a composition of the disclosure and/or to be used in the method of the disclosure can be determined by those skilled in the art, given the benefit of the present disclosure. Thus, in one embodiment, an effective amount of a composition of the disclosure is administered. An effective amount can be an amount of the composition that inhibits growth of cells in the individual, or an amount that extends the survival of the individual, or that alleviates disease symptoms associated with expression of the antigen in the individual, or enhances the effect of a chemotherapeutic agent to which cancer cells have developed resistance.

The method of the disclosure can be performed in conjunction with conventional therapies including but not limited to other chemotherapies, surgical interventions, and radiation therapy.

Cancers treated according to embodiments of this disclosure can be any type of cancer. In embodiments the cancer is a solid tumor which may be a solid tumor that is at risk for metastasis. In embodiments the individual may have a tumor that is at risk of or is undergoing metastasis. The individual may have previously had a metastatic tumor and is at risk for recurrence of a tumor and/or metastasis of it. In embodiments the cancer may be any one of fibrosarcoma, myxosarcoma, liposarcoma, chondrosarcoma, osteogenic sarcoma, chordoma, angiosarcoma, endotheliosarcoma, lymphangiosarcoma, pseudomyxoma peritonei, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, head and neck cancer, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilns' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oliodendroglioma, meningioma, melanoma, neuroblastoma, retinoblastoma, leukemia, lymphoma, multiple myeloma, thymoma, Waldenstrom's macroglobulinemia, and heavy chain disease.

In one embodiment the individual has or is at risk for developing ovarian cancer. In this regard, and without intending to be constrained by any particular theory, it is considered that the key biological processes leading to the formation of highly aggressive and metastatic ovarian cancer recurrences are not clearly understood, stressing the need for both an improved understanding of disease resistance as well as effective treatment options for relapsed cancers that are both phenotypically and biologically heterogeneous¹⁻³. Individual ovarian tumors show distinct sub-areas of proliferation and differentiation, often with regions undergoing epithelial-mesenchymal transition, where cancer initiating cells (CICs) have the capacity to indefinitely self-renew and sustain tumor growth⁴. It is thought that CD44⁺ CICs are able to survive conventional chemotherapies, giving rise to recurrent tumors that are more resistant and aggressive^{4,5}. The presence of CD44⁺ ovarian cancer cells has been correlated with chemoresistance to a front-line treatment with paclitaxel (PTX) and carboplatin (CBDCA) associated with induction of molecular modifications in the preexisting CICs^{6,7}. Given the heterogeneous nature of the ovarian tumor microenvironment (TME), therapeutic approaches that act across the different subtypes of epithelial ovarian cancer (EOC) and target both chemoresistant cancer cells and the TME that promotes tumor growth would be of clear benefit. Additionally, as the presence of tumor-infiltrating CD8⁺ T lymphocytes and a high CD8⁺/regulatory T cell (Treg) ratio are associated with improved survival in patients with ovarian tumors⁸⁻¹⁰, it is important that newly developed treatments also initiate or enhance antitumor immune responses that promote durable tumor control, which are improvements provided by the present disclosure.

Oncolytic viruses (OVs), including vaccinia (OVV), mediate anticancer effects by both direct oncolysis and stimulation of innate immune responses through production of damage-associated molecular patterns (DAMPs) and the presence of virus-derived pathogen-associated molecular patterns (PAMPs)^{11,12}, leading to increased type I IFN production^{13,14}. Additionally, OVV-mediated oncolysis may facilitate the direct acquisition of tumor-derived antigens by host antigen-presenting cells within the TME, thereby leading to improved T cell priming as well as coordination of the effector phase of antitumor immune responses. A currently initiated clinical trials of GL-ONC1 vaccinia virus against high-grade serous, endometrioid, or clear-cell ovarian cancer which includes: (1) platinum-resistant (recurrence or progression in <6 months) or (2) platinum-refractory (progression while on platinum-based therapy) (NCT02759588), emphasizes the unmet medical need to develop new therapies that are effective in patients that do not respond to chemotherapy.

In addition to a direct effect of oncolytic virotherapy on drug-resistant malignant cells, the interaction of cancer cells with their microenvironment, which protects the malignant cells from genotoxic stresses such as chemotherapy, is an attractive target to improve anti-cancer treatment. Several lines of evidence indicate that activation of the chemokine CXCL12 pathway increases tumor resistance to both conventional therapies and biological agents by: i) directly promoting cancer cell survival, invasion, and the cancer stem and/or tumor-initiating cell phenotype; ii) recruiting “distal stroma” (i.e., myeloid bone marrow-derived cells) to indirectly facilitate tumor recurrence and metastasis; and iii) promoting angiogenesis directly or in a paracrine manner^{15,16}.

It is expected that the anticancer efficacy can be greatly improved by inhibiting the CXCL12/CXCR4 axis. The present disclosure provides in non-limiting demonstrations analysis of the feasibility of targeting PTX- and CBDCA-resistant variants of murine ID8-R and human CAOV2-R ovarian cancer cells using the armed OVV [(i.e., expressing the CXCR4 antagonist in-frame with the Fc portion of murine IgG2a (OVV-CXCR4-A-Fc)] alone or in combination with doxorubicin (DOX). The latter drug was chosen for the combination treatment because the pegylated liposomal doxorubicin (PLD) has become a major component in the routine management of EOC used for treatment of platinum-resistant ovarian cancer¹⁷. Furthermore, as T-cell exclusion from tumors is associated with low expression levels of type IIFN associated genes¹⁸, increased expression of these genes during OVV infection may improve the impact of responses to anthracycline (i.e. DOX)-based chemotherapy¹⁹, and could potentiate the antitumor immune response by enhancing local infiltration of inflammatory cells following infection. Although DOX-based chemotherapy has been previously showed to synergize with oncolytic adenovirus against soft-tissue sarcomas in hamsters, the virus did not provide a clear advantage over DOX alone with regards to in vivo efficacy perhaps because the hamster model is only semi-permissive to human adenovirus²⁰. Using different delivery modes of OVV and DOX, we show that OVV delivered prior to DOX treatment elicited a multifaceted response resulting in a synergistic increase involving direct oncolysis of the resistant variants, a decrease in intratumoral recruitment of immunosuppressive elements, and stimulation of antitumor immunity that led to curative inhibition of tumor growth. These outcomes were most apparent following treatment with OVV-CXCR4-A-Fc, demonstrating that armed oncolytic virotherapy can further modulate the antitumor immune response.

The following Examples are intended to illustrate but not limit the disclosure.

### EXAMPLES

The following examples are intended to illustrate the disclosure. Those skilled in the art will recognized that minor modifications can be made without deviating from the spirit of the disclosure.

Increased Susceptibility of PTX- and CBDCA-Resistant Ovarian Tumor Cells to Vaccinia Virus Infection

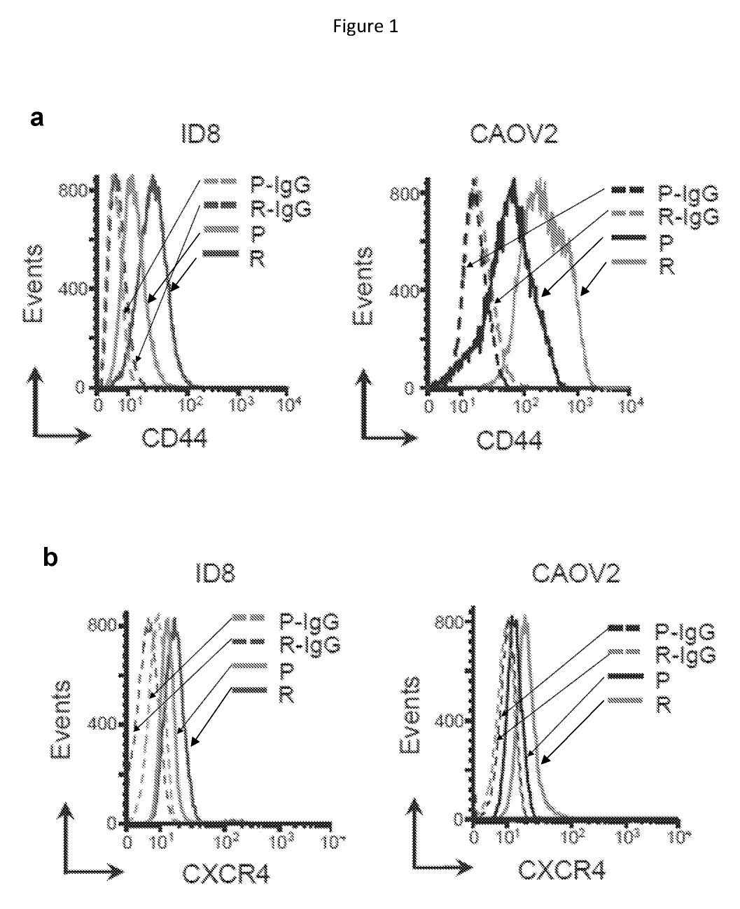

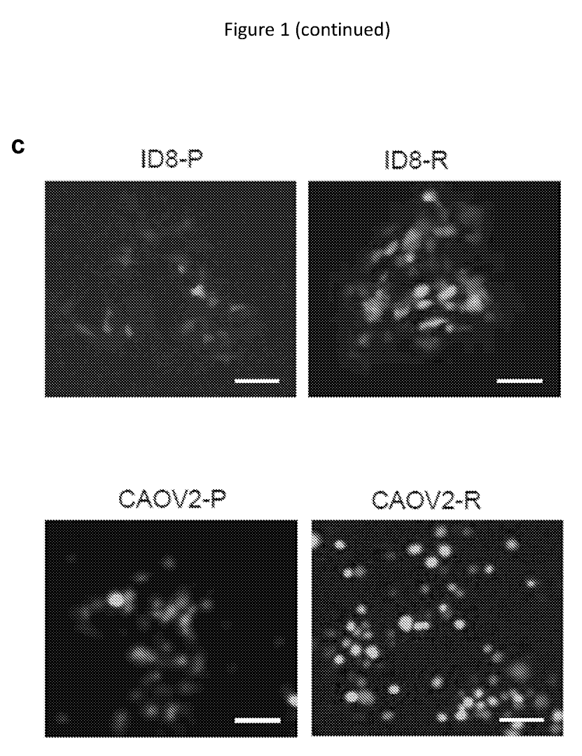

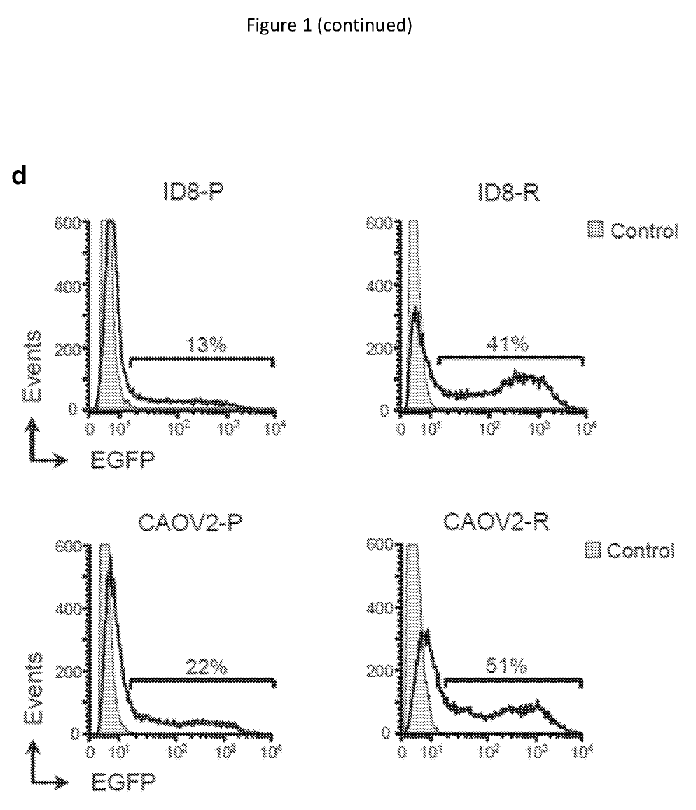

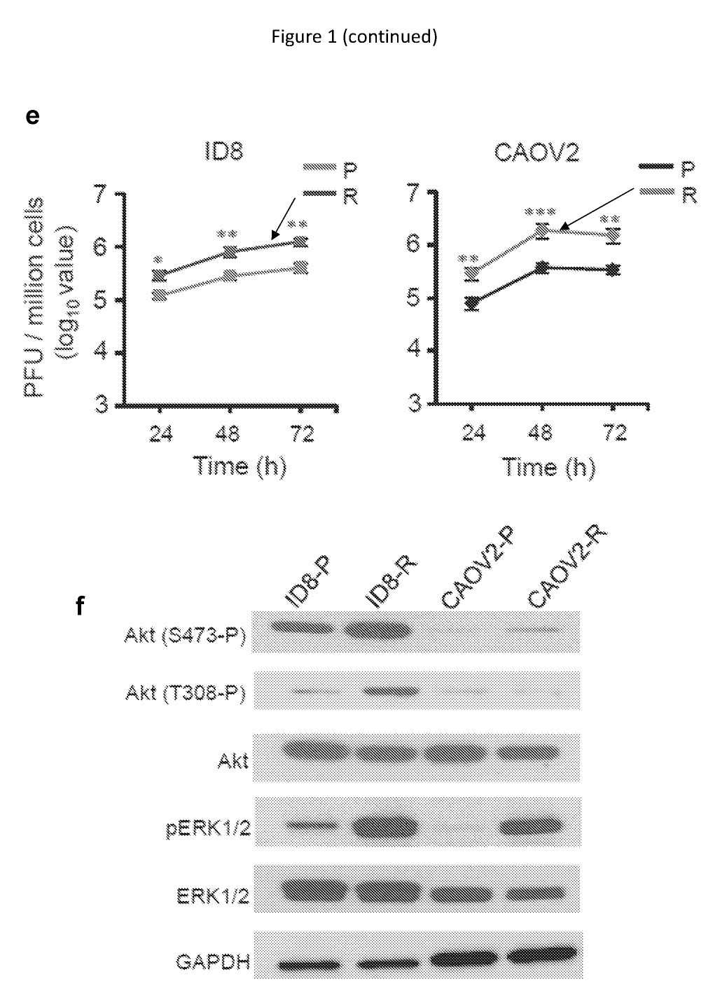

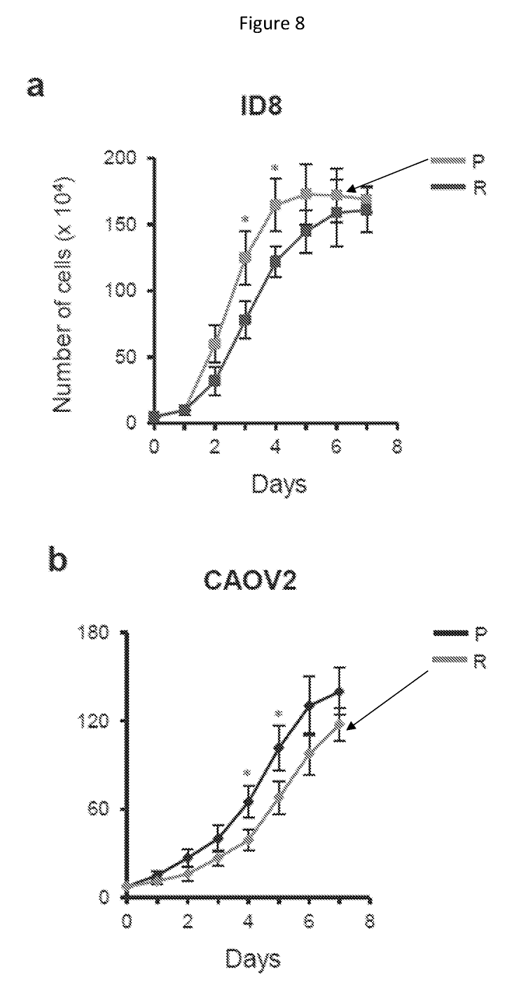

To investigate strategies for effective killing of drug-resistant ovarian tumor cell variants, we used PTX- and CBDCA-resistant murine ID8-R and human CAOV2-R ovarian tumors selected for drug resistance and maintained in media supplemented with PTX (59 nM) and CBDCA (2.6 μM). At these drug concentrations, the resistant variants exhibited small decreases in growth rates compared to their parental tumor cells (FIG. 8) and expressed over twofold higher levels of CD44 antigen in both ID8 (WI: 30±4 versus 12±2) and CAOV2 (WI: 225±15 versus 126±13) when compared to parental cell cultures (FIG. 1a). Expression of the CXCR4 receptor was also elevated in the resistant compared to the parental variants of ID8 (WI: 24±3 versus 11±1) and CAOV2 (WI: 36±5 versus 16±2) cells (FIG. 1b). Consistent with the increased CD44 and CXCR4 expression and their association with the ovarian CIC-like phenotype^{21,22}, ID8-R and CAOV2-R variants exhibited higher tumorigenicity when injected in exponentially smaller numbers intraperitoneally (i.p.) into syngeneic or SCID mice, respectively. As shown in Table 1, a minimum of 5×10⁶ ID8-P cells was required to consistently initiate i.p. tumor growth in all inoculated mice within a 8-wk period, whereas injection of 4% of that number formed ID8-R tumors within a much shorter period of time. Similar results were obtained with the parental and CAOV2-R tumors in SCID mice where the number of resistant cells necessary to form i.p. tumors was only 40% of the required number of parental cells (Table 1).

TABLE 1
In vivo tumorigenicity of parental and drug-resistant ID8 and
CAOV2 cells

| | Injection | | | Tumor | Latency |
|---|---|---|---|---|---|
| Cell type^{a} | route | Mice | Cell dose^{b} | formation^{c} | days^{d} |
| ID8-P | i.p. | C57BL/6 | 5 × 10⁶ | 5/5 | 60.4 ± 4.2 |
| ID8-P | i.p. | C57BL/6 | 1 × 10⁶ | 1/6 | 72 |
| ID8-P | i.p. | C57BL/6 | 5 × 10⁵ | 0/5 | NA^{e} |
| ID8-R | i.p. | C57BL/6 | 1 × 10⁶ | 6/6 | 24.1 ± 4.5 |
| ID8-R | i.p. | C57BL/6 | 2 × 10⁵ | 8/8 | 29.4 ± 3.6 |
| ID8-R | i.p. | C57BL/6 | 1 × 10⁵ | 4/6 | 38.3 ± 3.1 |
| ID8-R | s.c. | C57BL/6 | 5 × 10⁴ | 0/5 | NA |
| CAOV2-P | i.p. | SCID | 5 × 10⁶ | 5/5 | 37.4 ± 4.8 |
| CAOV2-P | i.p. | SCID | 3 × 10⁶ | 3/6 | 59.8 ± 3.7 |
| CAOV2-P | i.p. | SCID | 1 × 10⁶ | 1/5 | 82 |
| CAOV2-R | i.p. | SCID | 2 × 10⁶ | 8/8 | 29.2 ± 6.1 |
| CAOV2-R | i.p. | SCID | 1 × 10⁶ | 4/5 | 46.3 ± 9.2 |
| CAOV2-R | i.p. | SCID | 5 × 10⁵ | 1/5 | 73 |

^{a}Parental and PTX- and CBDCA-resistant ID8 and CAOV2 tumor cells were injected i.p. at different numbers into syngeneic C57BL/6 or SCID mice and monitored for tumor growth.
^{b}No. of cells/injection.
^{c}No. of tumors/no. of injections.
^{d}Time from injection to the first appearance of ascites. Results are presented as mean □□SD.
^{e}NA, not applicable.

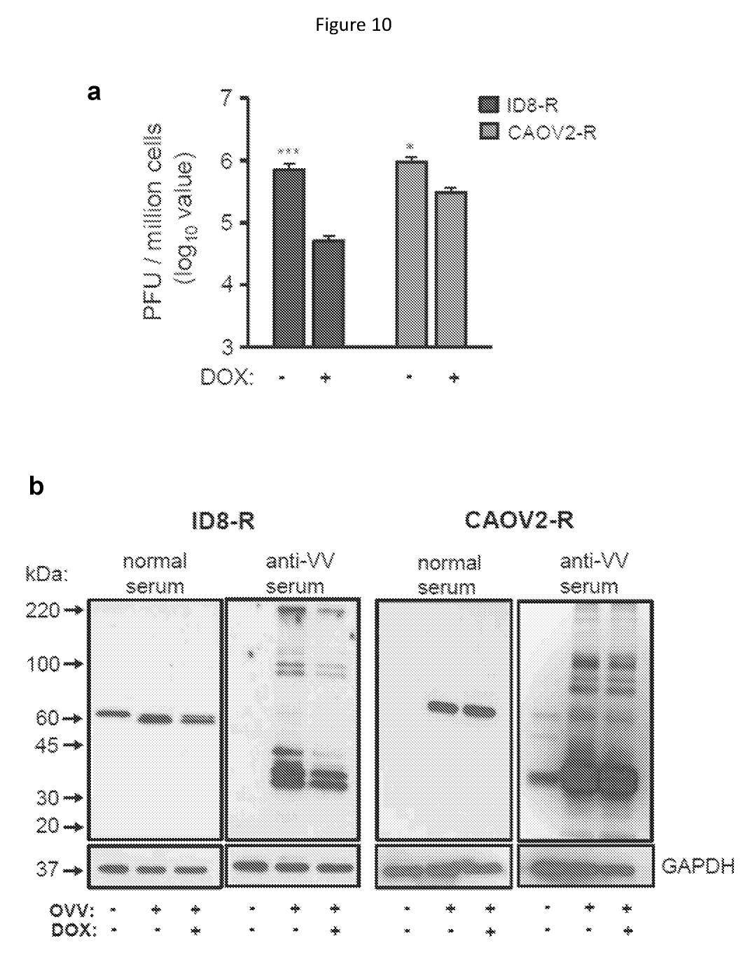

Infection of the resistant variants with oncolytic vaccinia virus expressing the enhanced green fluorescence protein (OVV-EGFP) at multiplicity of infection (MOI) of 1, showed over 2-fold increase in the number of EGFP⁺ cells 24 h after infection (FIGS. 1c and 1d) and resulted in higher viral titers than those recovered in the parental cultures (FIG. 1e, P≤0.01). In line with evidence that Akt²³ and MEK/ERK²⁴ pathways augment vaccinia replication²⁵, Western blotting of ID8-R cellular lysates revealed 2- and 6-fold higher Akt phosphorylation levels at S473 and T308 as well as 6-fold higher ERK1/2 phosphorylation compared to the parental cells (FIG. 10. In CAOV2-R variants, the level of Akt(S473-P) showed small increases in contrast to 26-fold higher expression of pERK1/2 compared to parental cells (FIG. 1f).

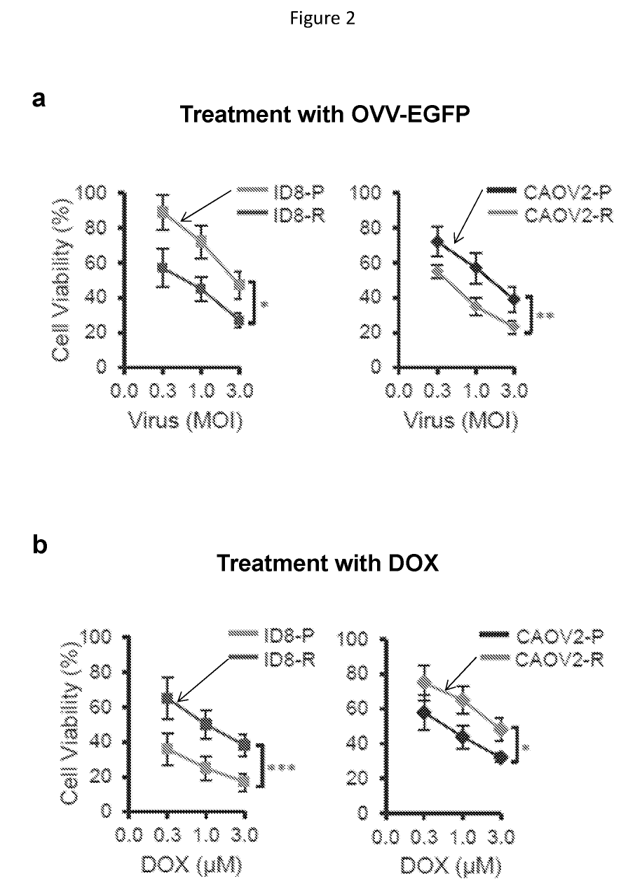

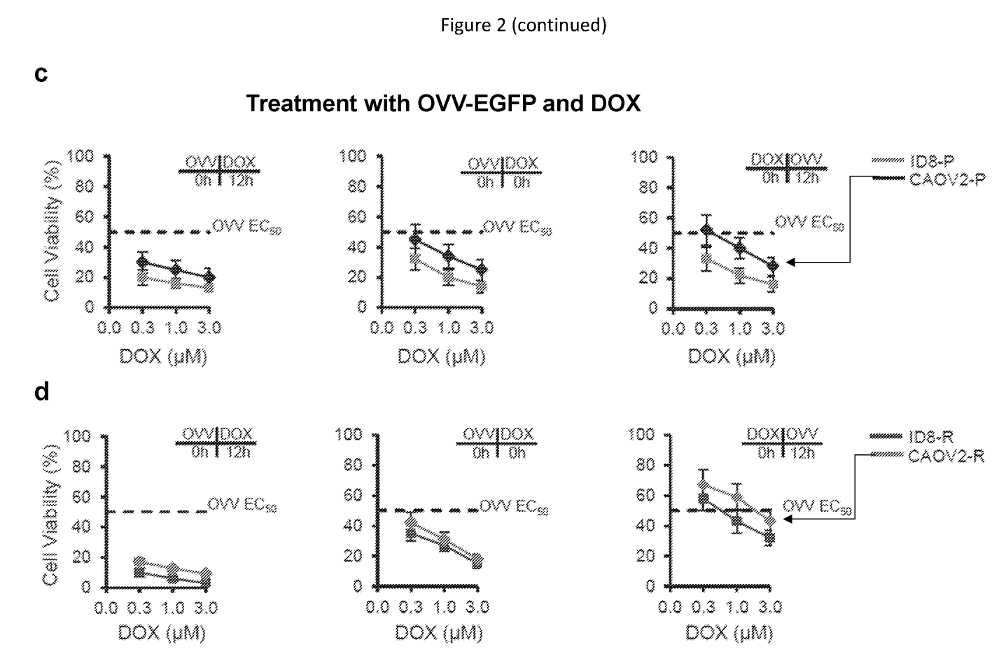

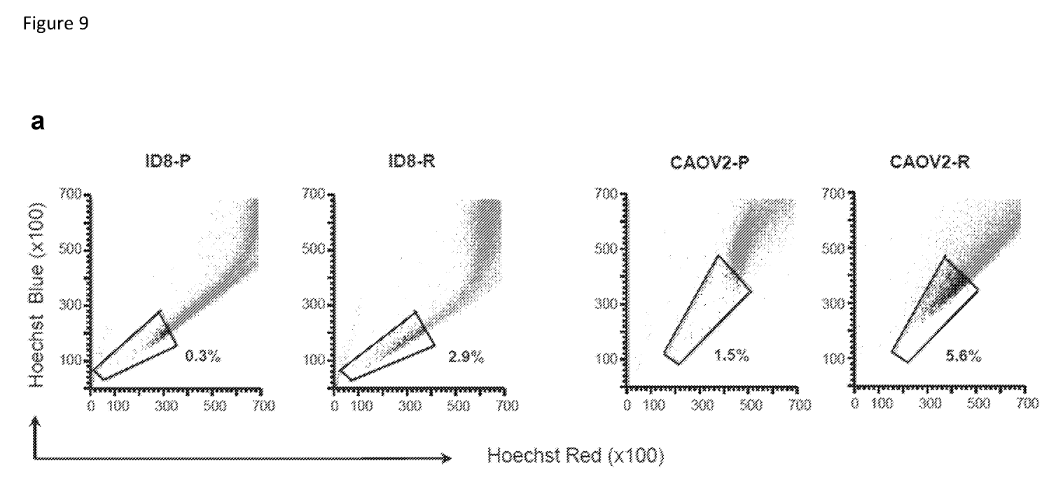

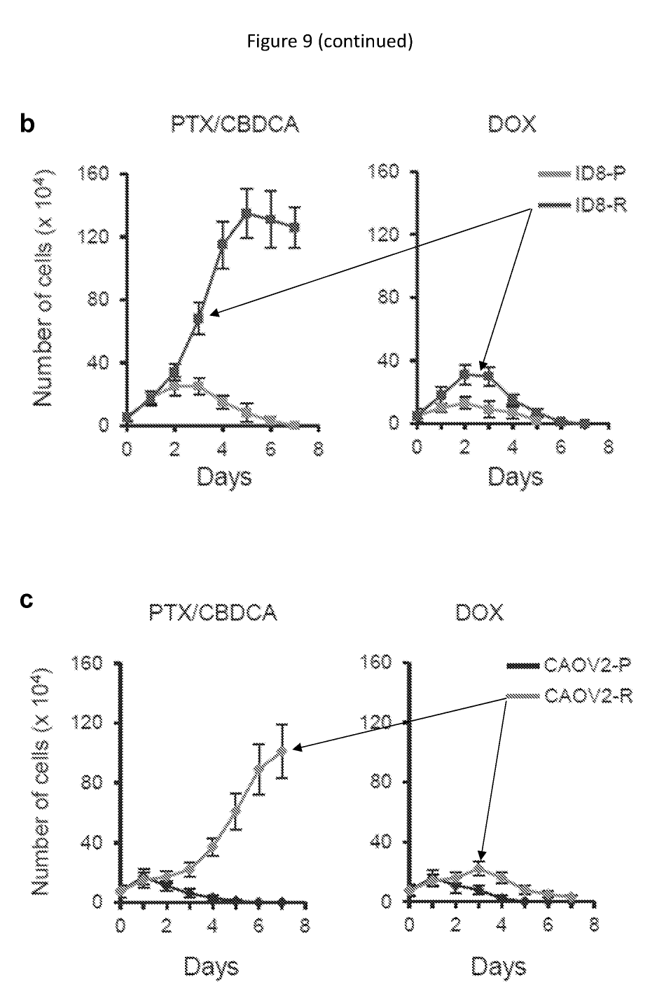

Cytopathic effects of OVV and DOX combination treatments. We next examined whether cytopathic effects of vaccinia virus could be augmented by DOX treatment using a 72-h cell viability assay with serial dilutions of OVV-EGFP and DOX added alone or in combination to the parental and drug-resistant variants of ID8 and CAOV2 cells. As expected based on the higher infection and replication rates of vaccinia, ID8-R variants were more susceptible to the lytic effect of vaccinia than the parental cells based on 5-fold less virus needed to achieve 50% killing (EC₅₀) (FIG. 2a, left panel). Similarly, CAOV2-R cells were 4-fold more sensitive to the vaccinia-mediated killing than the parental cells (FIG. 2a, right panel). However, the sensitivity profile of drug-resistant variants to DOX-mediated killing was opposite to that of the virus. For example, the IC₅₀ values for ID8-R cells were approximately 10-fold higher compared to the parental cells (P=0.0003; FIG. 2b, left panel). The increased resistance to DOX was also evident in CAOV2-R cells (FIG. 2b, right panel) with 3-fold differences in the ICso values between the resistant and parental cells (P=0.04). The increased cross-resistance of ID8-R and CAOV2-R variants compared to their parental counterparts could be attributed to higher proportions of the “side population” (SP) cells in the resistant variants whose intrinsic dye efflux export many cytotoxic drugs and enhance resistance to chemotherapeutic agents^{26,27}. Staining with fluorescence dye Hoechst 33342 showed 3- to 5-fold higher proportion of Hoechst^{low} SP cells in ID8-R and CAOV2-R cells compared to the parental cells (range: 0.3%-1.5% versus 2.9%-5.6%; FIG. 9a). Culturing the tumor cells in the presence of DOX (3 μM) showed that the initial growth rates of the resistant variants in the presence of DOX were lower compared to those in PTX (59 nM) and CBDCA (2.6 μM) with over 90% dead cells after a one-week period (FIG. 92b,c), indicating that increases in SP cells were not adequate to afford a durable survival advantage in the presence of DOX.

Because both the parental and the resistant cell lines were susceptible to the cytopathic effect of OVV and DOX, albeit with different levels of efficacy, we next asked whether these two therapies could potentially synergize to further enhance tumor cell killing. To determine whether the sequence of the treatments was important, the parental and resistant cells were treated with different concentrations of DOX added 12 h after, simultaneously, or 12 h before the virus used at the EC₅₀ titers. As shown in FIG. 2c,d (left panel), treatment of both variants with vaccinia for 12 h prior to DOX revealed the highest cytopathic effect compared to monotherapy treatments. However, the effect was less prominent in the parental cells than in the resistant variants reflecting the low susceptibility of these cells to vaccinia infection and high sensitivity to DOX. On the other hand, −80% of cell death was achieved in both ID8-R and CAOV2-R cultures even at concentrations of DOX that had small cytopathic effects when used alone, indicating a synergistic interaction between these two agents in cell-mediated killing of resistant cells. The simultaneous treatment with both agents appeared to be less than additive (FIG. 2c,d, middle panel), whereas treatment of tumor cells with DOX prior to infection inhibited viral killing (FIG. 2c,d right panel). Thus, the over 5-fold difference in cytopathic effects between the most and least effective combination treatments (e.g., OVV followed by DOX versus DOX followed by OVV) in the resistant cultures compared to only 2-fold difference in the parental counterparts suggest that the chemosensitivity profile of tumor cells affects efficacy of the OVV and DOX delivery. The reduced viral replication in the presence of DOX is consistent with 10- and 3-fold decreases in viral titers in ID8-R and CAOV2-R cultures treated with DOX 12 h following vaccinia infection compared to virus infection alone (FIG. 10a). Also, Western blotting of the infected and DOX-treated cultures with vaccinia-specific serum revealed lower expression of viral antigens compared to cells infected in the absence of the drug with more prominent differences in ID8-R cells (FIG. 10b).

Protection Against ID8-R Metastases by Single and Combination Treatment with OVV and PLD

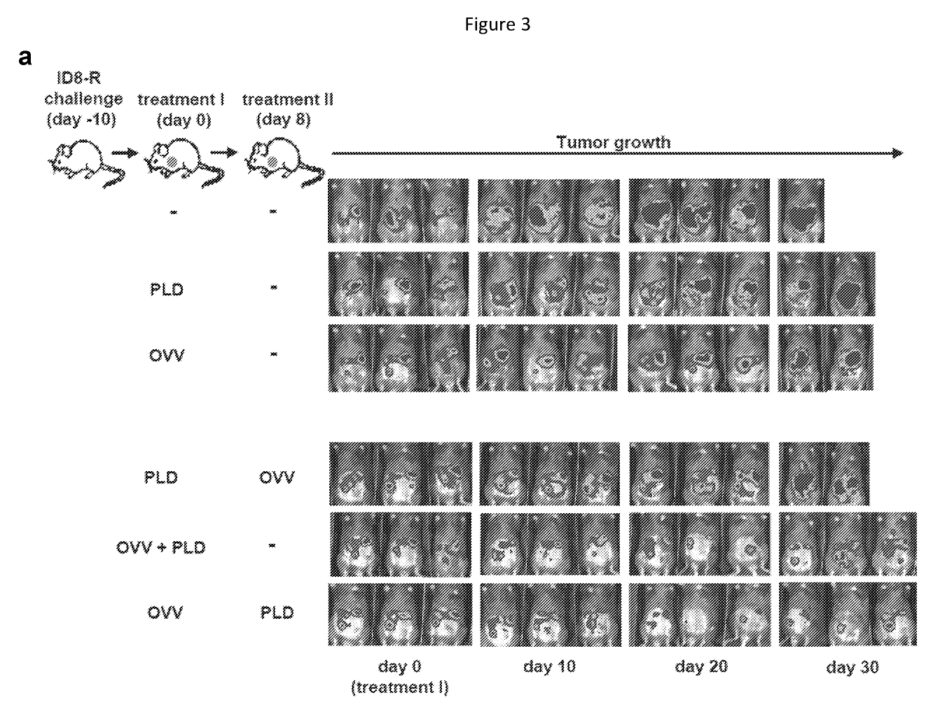

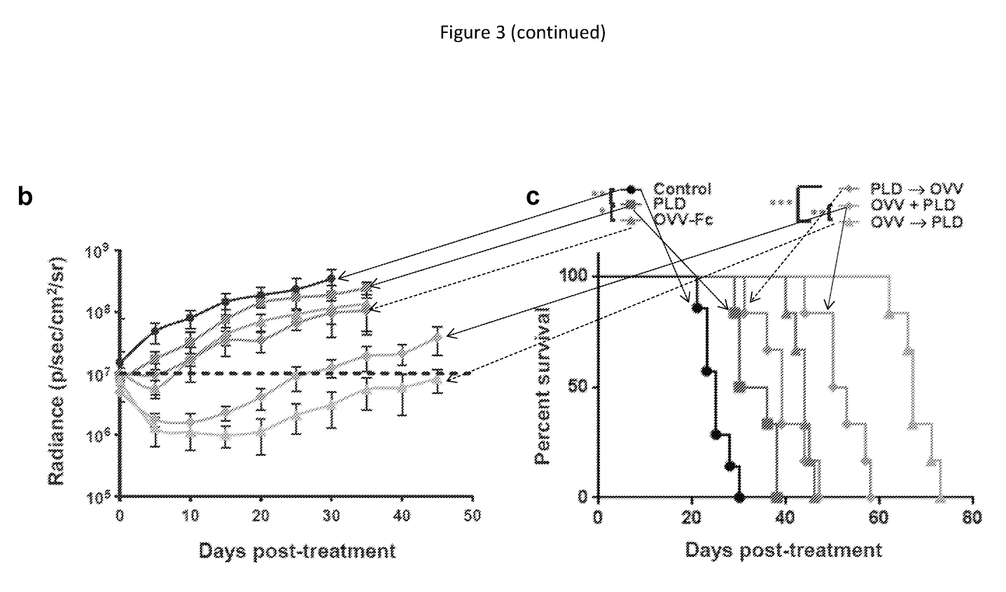

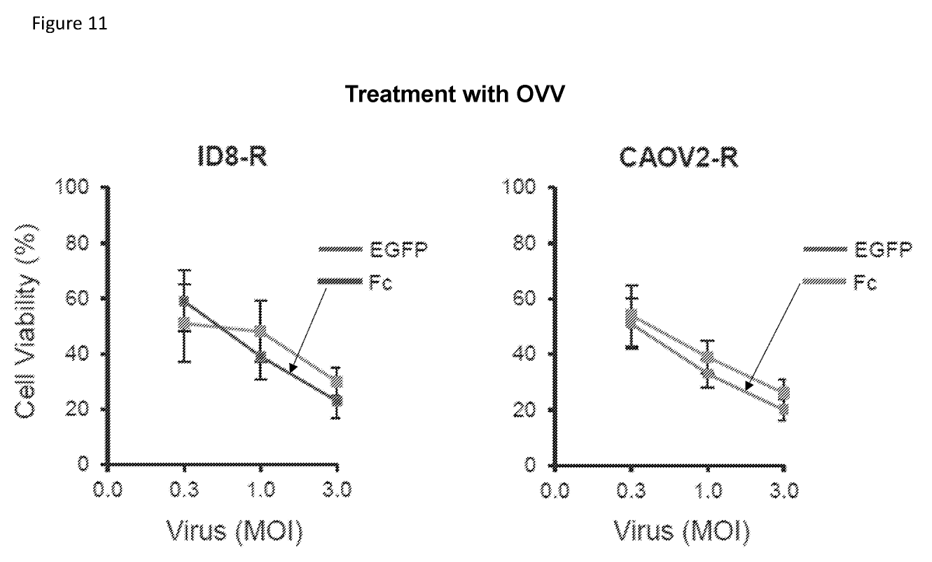

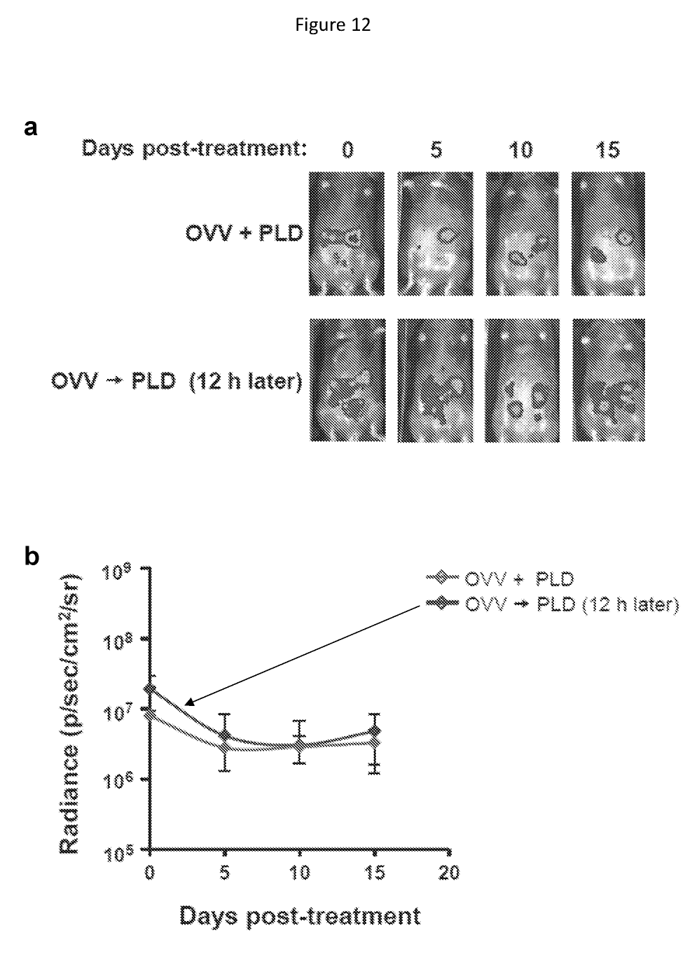

To effectively test the multiple mechanisms of synergy between OVV and DOX treatments, we next examined whether the effect of DOX on tumor cell killing in vitro could be translated to the orthotopic growth of ID8-R and CAOV2-R tumors in syngeneic and SCID mice, respectively. For the in vivo experiments, DOX was replaced with its pegylated liposomal form known as PLD whereas OVV-EGFP was replaced with the Fc portion of murine IgG2-expressing vaccinia virus (OVV-Fc) because the immune response against EGFP in infected mice could alter the treatment efficacy²⁸. Both OVV-EGFP and OVV-Fc have similar effects on tumor cell killing (FIG. 11). ID8-R cells (2×10⁵) were injected i.p. into syngeneic C57BL/6 mice and treated 10 days later with PLD or OVV-Fc delivered as single agents or in combination. PLD (10 mg/kg) was delivered i.v. whereas vaccinia viruses (10⁸ PFU/injection) were delivered i.p. Because the kinetics of vaccinia virus spreading infection in tumor-bearing mice differs from that in cell cultures, with the peak and cessation of viral replication occurring on days 4 and 8, respectively^{29,30}, we used an 8-day interval period between OVV-Fc and PLD treatments to ensure that PLD did not interfere with vaccinia replication (FIG. 3a). Progression of tumor growth, quantified by bioluminescence imaging (FIG. 3a,b), revealed rapid tumor progression in control mice that were euthanized within five weeks after tumor challenge (FIG. 3c). PLD treatment alone was not effective in controlling tumor spread and extended survival by approximately one week compared to the control mice. The antitumor efficacy of OVV-Fc or OVV-Fc delivered after PDL reduced progression of tumor growth by two weeks after which period tumor growth continued at a rate similar to that in the control mice. Treatment with OVV-Fc followed by PLD had more potent antitumor activities extending the slower rate of tumor growth and survival for approximately four and two weeks compared to mice treated with the virus only (P<0.001) or virus delivered together with PLD (P=0.002; FIG. 3c). Interestingly, the antitumor effect of OVV-Fc and PLD treatments delivered together was similar to that achieved when PLD was delivered 12 h after the viral infection (FIG. 12), highlighting that a longer time interval between delivery of the two treatments is required to achieve optimal therapeutic benefit.

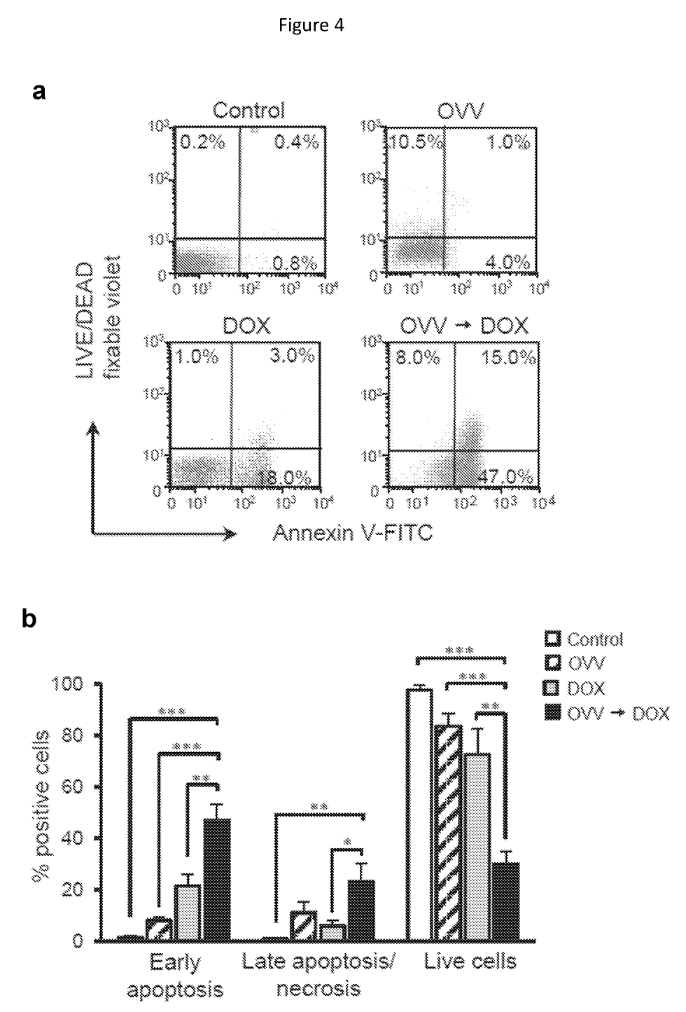

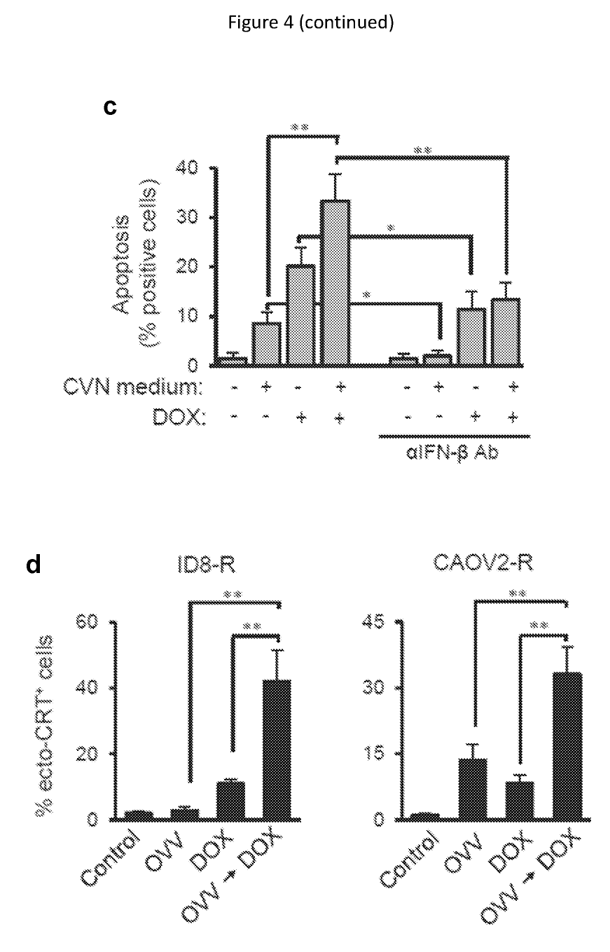

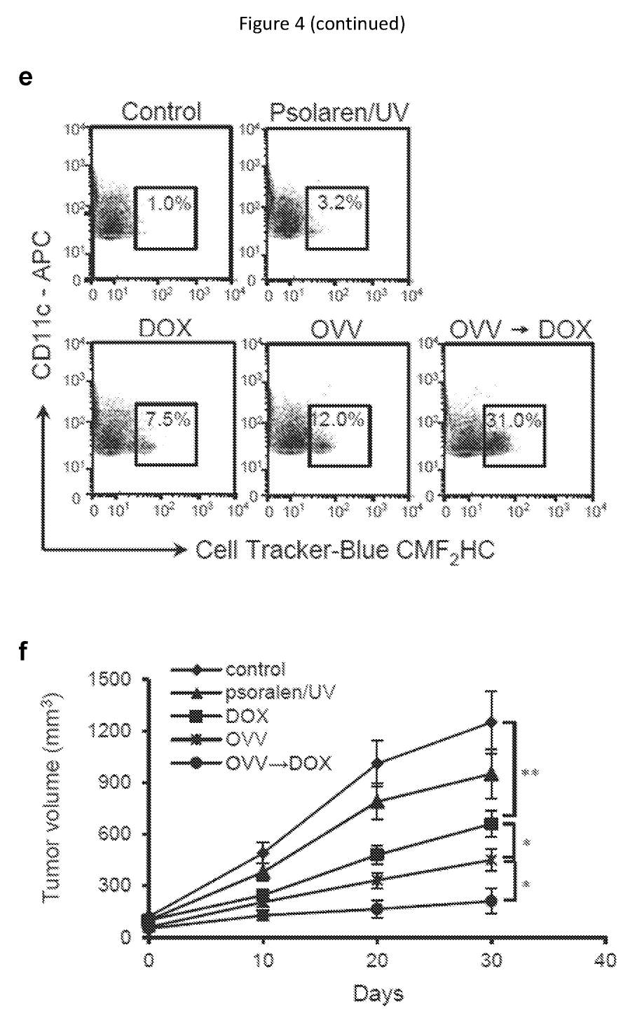

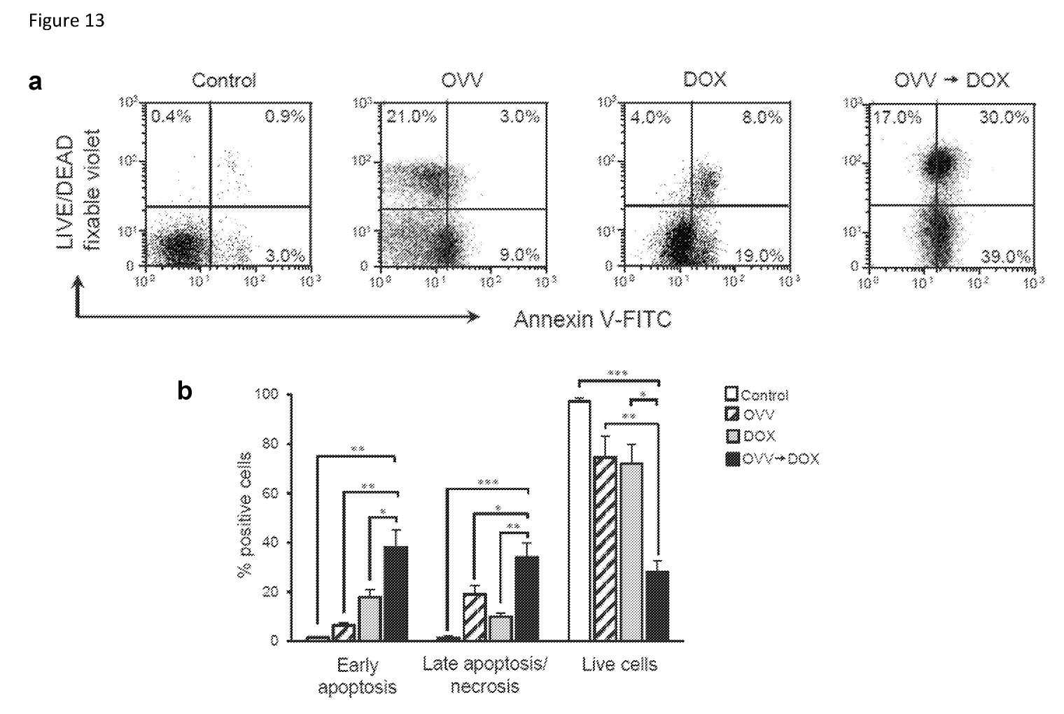

Immune responses against dying cells after OVV followed by DOX treatment are associated with increased apoptosis and phagocytosis of tumor cells by DCs. To explore cellular mechanisms involved in the synergistic killing of the drug-resistant tumor cells with vaccinia followed by DOX treatment, we investigated the induction of tumor cell death by each treatment alone or in combination. The induction of apoptosis and necrosis was investigated in 24 h cultures of ID8-R tumor cells treated with vaccinia virus at MOI of 1, which roughly corresponds to the EC₅₀ titer, or DOX (1 μM) alone or in combination. In the combination treatment, OVV was added 12 h before DOX and induction of apoptosis/necrosis was analyzed by flow cytometry with Annexin V-FITC and LIVE/DEAD fixable violet. While DOX or vaccinia alone induced apoptosis or necrosis in ˜20% of cells, the combination treatment significantly increased early apoptosis (Annexin V⁺ and LIVE/DEAD fixable violet⁻) compared to cultures treated with OVV-Fc or DOX only (FIG. 4a,b; P=0.0008 and P=0.009). Late apoptosis/necrosis (Annexin V^{+/-} and LIVE/DEAD fixable violet⁺) was increased to a lesser extent, altogether reducing the number of viable cells by 70%. Similar results were observed with CAOV2-R cells (FIG. 13). To address the possibility that the viral infection produced a bystander effect by releasing factors capable of sensitizing ID8-R tumor cells to DOX, culture supernatants from uninfected or 24 h-infected ID8-R cultures were filtered and treated with UV and psolaren to remove any infectious virus prior to adding to fresh uninfected cells. OVV treatment-conditioned and virus-negative (CVN) medium, when added to uninfected ID8-R cultures, induced modest increases in early apoptosis but significantly enhanced the effect of DOX (FIG. 4c; P=0.03). This effect could be attributed to IFN-β production which was detected in the CVN medium by ELISA (86±13 pg/ml). Treatment with IFN-β blocking antibody reduced the apoptotic activity of CVN medium alone, or when combined with DOX, by as much as 80% (FIG. 4c). This finding supports the previously reported ability of type IIFN to enhance anthracycline-based Chemotherapy®. The viral treatment of ID8-R or CAOV2-R cells followed by DOX was also effective in inducing surface exposure of calreticulin (ecto-CRT) (FIG. 4d) known to enhance immunogenicity of cancer cell death³¹.

In view of the established role of surface CRT as an “eat me” signal^{32,33}, we investigated the phagocytosis of the treated tumor cells by bone marrow (BM)-derived DCs, which is stringently required for mounting immune response against dying tumor cells³⁴. As shown in FIG. 4e, ID8-R tumor cells that received the combination of OVV and DOX were over 3-fold more efficiently phagocytosed by DCs compared to either agent alone. To test the immunogenicity of tumor cells treated with single agent or combination therapies as vaccines, ID8-R cells exposed to OVV-Fc or DOX alone or in combination were injected into one flank of immunocompetent C57BL/6 mice. The mice were then challenged with live ID8-R cells injected into the opposite flank 8 days later. Protection against tumor growth was interpreted as a sign of successful vaccination and induction of antitumor immunity (FIG. 4f), since such protection was not observed in SCID mice (data not shown). These data suggest that the combination of OVV and DOX led to upregulation of factors associated with immunogenic cell death (ICD) that could potentiate the benefits of direct tumor cell killing by augmenting the induction of antitumor immunity.

Inhibition of i.p. Dissemination of ID8-R Tumor and Improved Overall Survival by OVV-CXCR4 Followed by PLD Treatment.

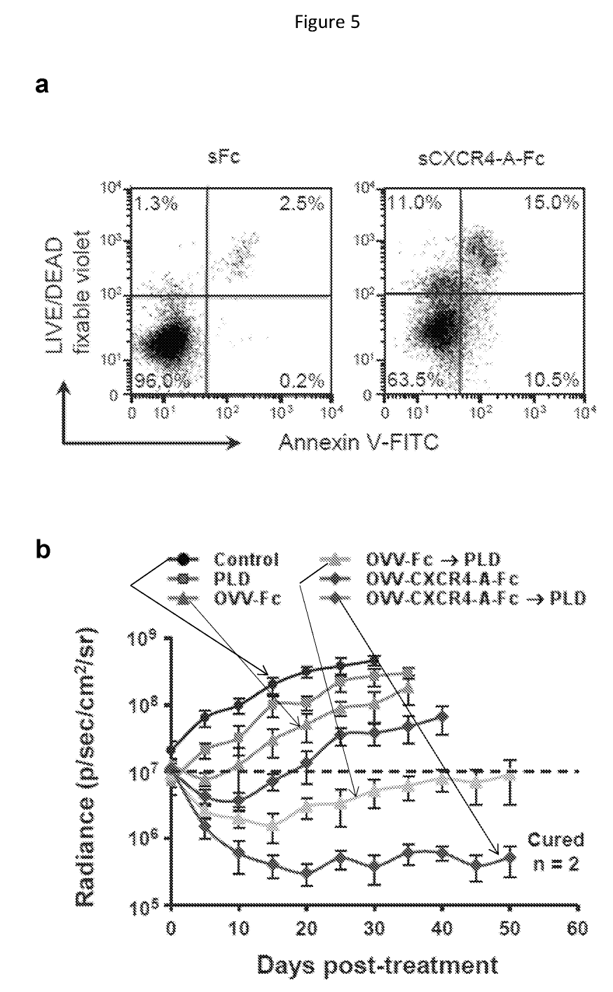

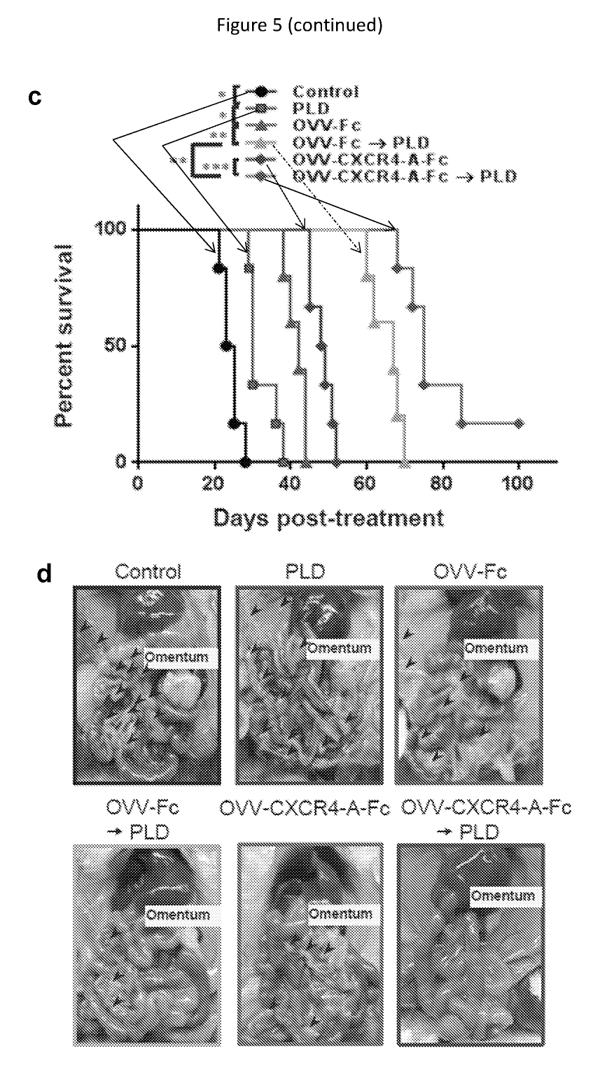

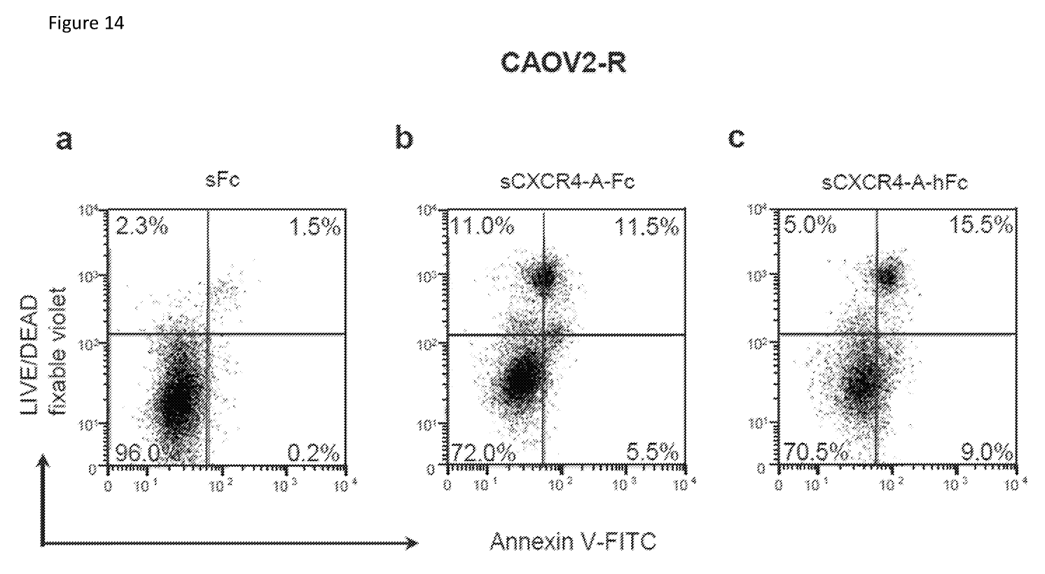

Although the combination treatment with OVV-Fc followed by PLD significantly inhibited growth of ID8-R tumor in vivo compared to single modality treatments, it did not provide permanent regression. This, together with the accumulating evidence that the chemokine CXCL12 pathway increases tumor resistance to both conventional therapies and biological agents^{15,16,29,35,36}, prompted us to employ an armed virus expressing a CXCR4 antagonist. The antagonist, developed based on the CTCE-9908 peptide analog of CXCL12^{37,38} and expressed in the context of murine (Fc) or human (hFc) fragment of IgG²⁹, is capable of binding and inducing apoptosis in ˜30% of CXCR4-expressing ID8-R and CAOV2-R cells (FIG. 5a and FIG. 14). We next examined whether a targeted delivery of CXCR4-A-Fc by the virus followed by PLD would lead to improve overall survival of syngeneic mice challenged i.p. with ID8-R tumors. OVV-Fc was used as a control for these studies. Progression of tumor growth quantified by bioluminescence imaging revealed that although each monotherapy decreased tumor growth and metastatic dissemination compared to untreated controls, no single agent treatments alone eliminated tumors (FIG. 5b,c). Treatment with OVV-Fc followed by PLD had more potent antitumor activities extending the slower rate of tumor growth and survival for almost four weeks compared to mice treated with the virus or PLD only (P<0.006). However, oncolytic virotherapy using the armed OVV-CXCR4-A-Fc virus followed by PLD was most effective in inhibiting tumor growth resulting in tumor-free survival in ˜20% of ID8-R tumor-bearing mice (FIG. 5b,c). In ID8-R tumor-bearing mice receiving the combined treatment with the armed virus, tumor growth was localized primarily in the omentum with only sporadic metastatic nodules (>5 mm) present in the peritoneal cavity (FIG. 5d). The metastatic spread of the tumor was more prominent after OVV-Fc than the OVV-CXCR4-A-Fc treatment. In contrast, the control mice or those treated with PLD had metastatic nodules present on the omentum, mesentery, diaphragm, and peritoneal wall.

OVV-CXCR4-A-Fc Followed by PLD Inhibits Tumor-Immunosuppressive Networks and Induces Antitumor CD8⁺ T Cell Responses

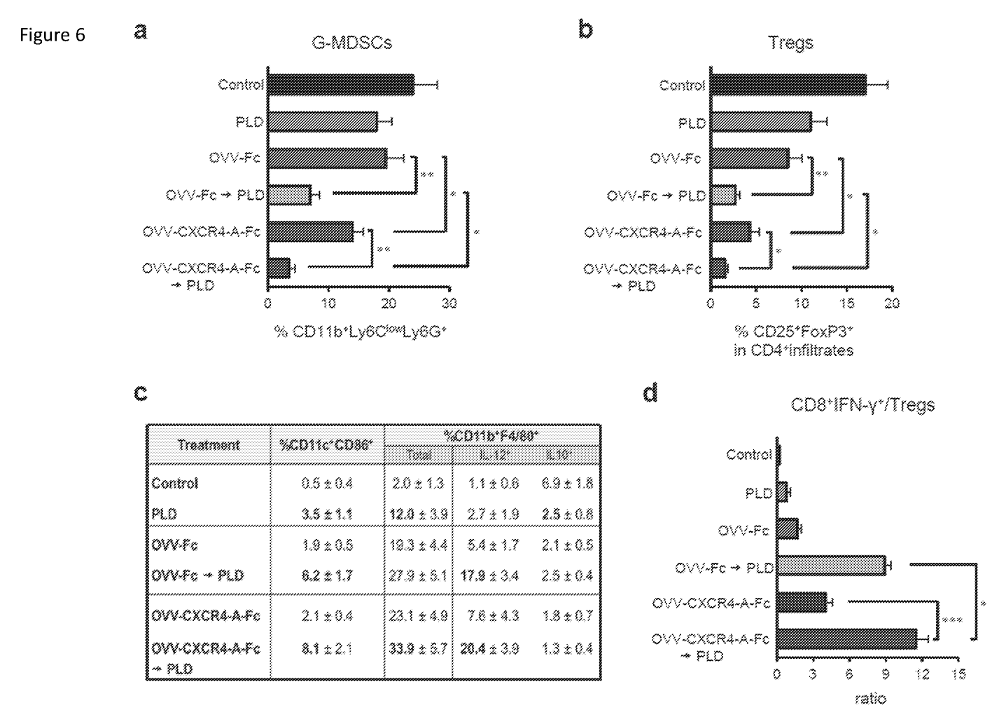

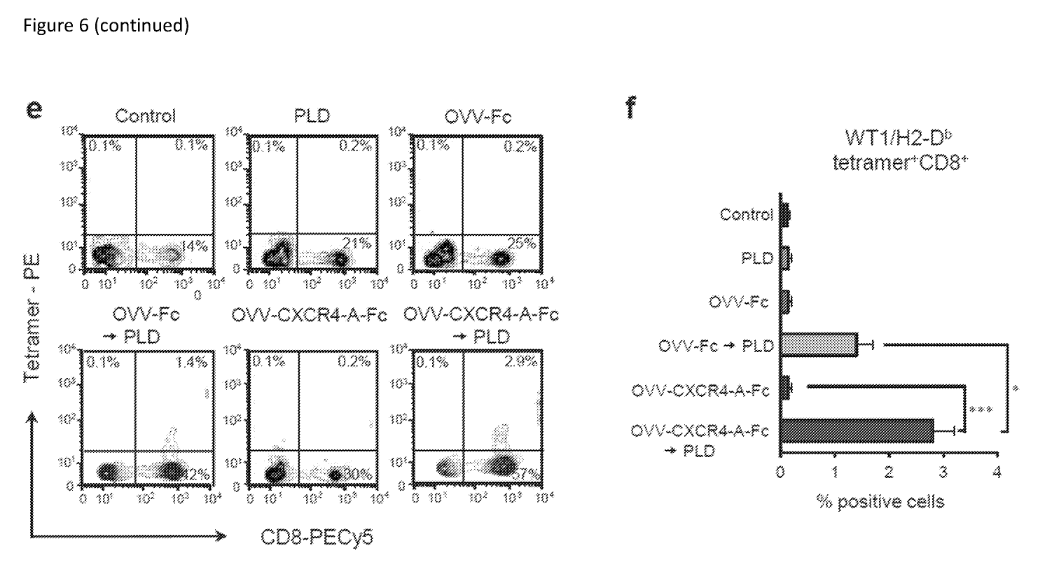

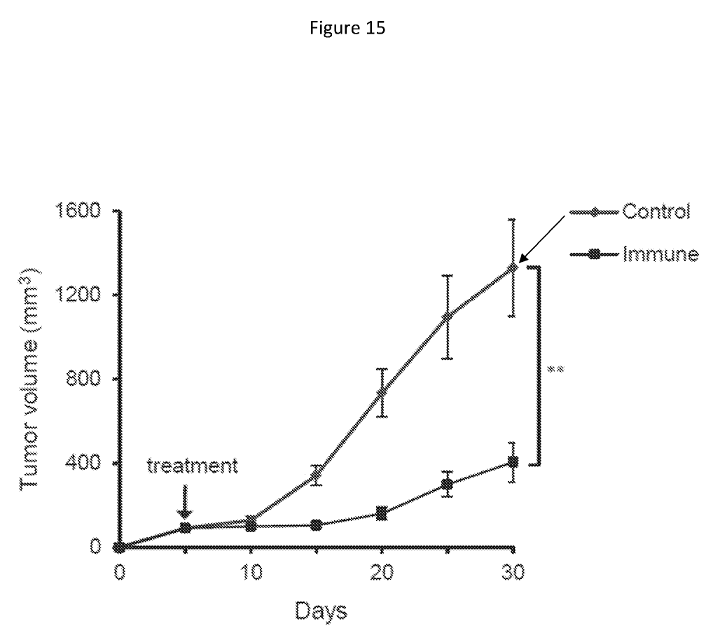

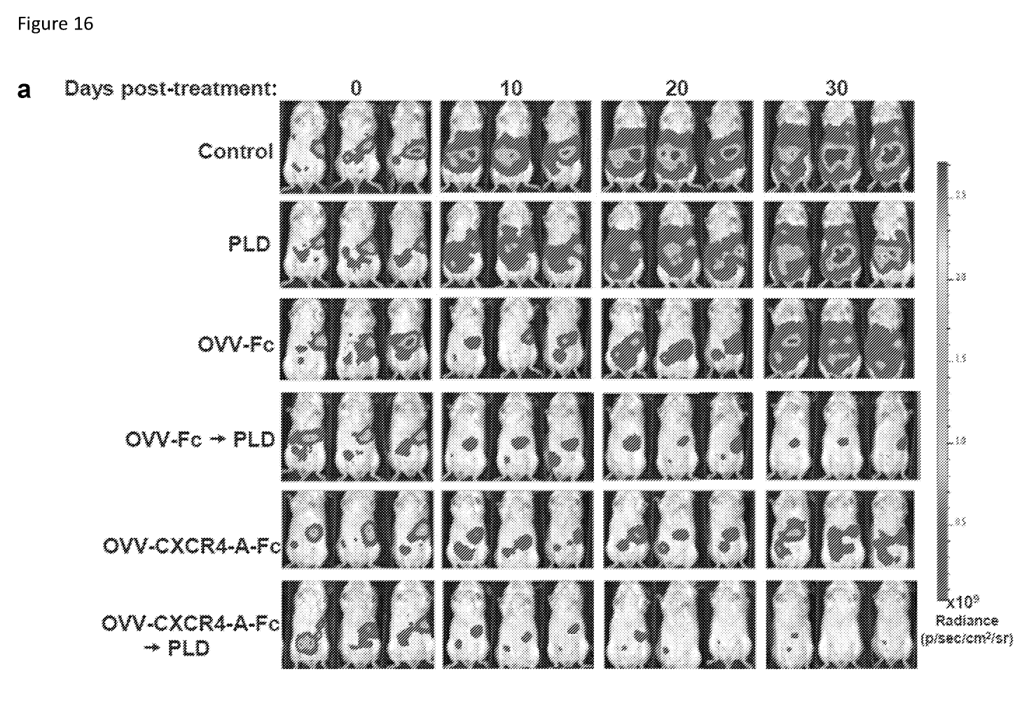

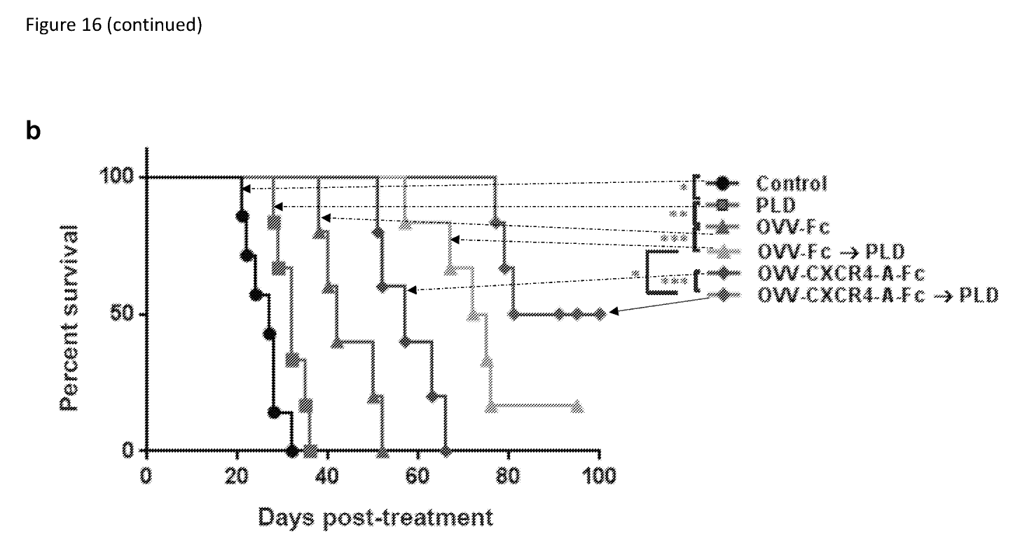

We next investigated the effect of the single and combination treatments on intratumoral accumulation of neutrophils/granulocytic myeloid-derived suppressor cells (G-MDSCs) and Tregs^{29,36} within the TME. The analysis performed on day 8 after completion of treatments revealed that the inhibition of tumor growth in ID8-R-bearing mice was associated with reduction of intraperitoneal recruitment of G-MDSCs (CD11⁺Ly6G^{high}Ly6C^{low}) and Tregs (CD4⁺CD25⁺Foxp3⁺) (FIG. 6a,b). Strikingly, the combination treatment resulted in increased frequencies of CD11c⁺CD86⁺DCs and IL-12-producing CD11b⁺F4/80⁺ inflammatory monocytes/macrophages in the peritoneal fluids of tumor-bearing mice (FIG. 6c). These changes were associated with higher ratios of IFN-γ-producing CD8⁺ to Tregs in the tumor-bearing animals treated with OVV-Fc or OVV-CXCR4-A-Fc followed by PLD as well as the presence of infiltrating tumor-specific CD8⁺ T cells specific for the Wilms' tumor antigen 1 (WT1), a clinically relevant antigen target⁴¹ expressed by ID8-R cells (FIG. 6d-f). We then adoptively transferred 2×10⁷ splenocytes from tumor-free mice with detectable WT1-specific T cell responses to ID8-R-bearing mice after combining them with LPS-matured WT1₁₂₆₋₁₃₄ peptide-pulsed BM-derived DCs⁴². Mice receiving splenocytes from animals with WT1-specific T cell responses showed reduced tumor growth compared to control mice, indicating the ability of the combined OVV-CXCR4-A-Fc and PDL treatment to promote the generation of durable antitumor immune responses (FIG. 15). Similarly, tumor-free survival was observed in 10% and 50% of CAOV2-R-bearing SCID mice treated with OVV-Fc and PLD and OVV-CXCR4-A-Fc and PLD, respectively (FIG. 16a,b). These results could be attributed to longer duration of productive viral replication/oncolysis in SCID mice and a direct effect of the CXCR4 antagonist on tumor cells as well as complement-dependent and antibody-dependent cell-mediated cytotoxicities²⁹.

Cancer cells, with a high propensity for mutation, allow drug-resistant clones to emerge in tumors after anti-cancer drug therapy. This process, combined with the ability of tumors to influence their microenvironment by subverting stromal cells, culminate in treatment resistance, tumor relapse, and therapy failure⁴³, suggesting that treatment strategies that can engage the patients' immune defense mechanisms through induction of ICD are important in contemporary cancer therapy. The approval of PLD in 1999, the recent FDA-approval of talimogene laherparepvec (T-VEC) virotherapy, and the ongoing clinical trial NCT02759588 of GL-ONC1 vaccinia virus against platinum-resistant and refractory ovarian cancers, all indicate we are entering a phase where these agents may significantly boost the armamentarium for cancer treatment. Importantly it is clear that platinum-resistant tumors become resistant to PLD used as a second line of treatment⁴⁴. Also, oncolytic viruses are eliminated through induction of anti-viral immune responses. Therefore, an effective combination treatment requires a well-coordinated strategy that would i) synergistically augment tumor cell killing with simultaneous induction of ICD, ii) reduce intratumoral recruitment of immunosuppressive elements in favor of immunostimulatory signals (i.e., IL-12), and iii) enhance local tumor-specific T cell accumulation to overcome a non-T-cell-inflamed TME to induce potent and durable antitumor immune responses. In this disclosure, we have demonstrated that ICD-inducing combination treatment consisting of OVV-CXCR4-A-Fc followed by PLD in PTX- and CBDCA-resistant ovarian tumor-bearing syngeneic mice significantly increased overall survival compared to single treatment modalities and reversed the immunosuppressive phenotype of the TME while promoting antitumor immunity.

Vaccinia virus can be considered as a suitable oncolytic virus candidate for treatment of drug-resistant ovarian tumors owing to its ability to infect a broad range of cells including CICs^{36,45}, a rapid replication cycle, production of extracellular enveloped virions that evade the immune response⁴⁶, and a capacity to spread to distant metastases following local delivery⁴⁷. However, replication of the virus in CD44-expressing drug-resistant variants has not been systematically explored and the mechanisms by which viral infection and replication is increased in resistant cells are still unclear. Vaccinia entry into target cells is thought to be mediated by glycosaminoglycans (GAGs) such as cell surface heparan sulfate (HS) that interacts with A27L viral membrane protein involved in a fusion of the virus to infected cells^{48,49}. As isoforms of CD44 are differentially modified by GAGs including HS, chondroitin sulfate, and dermatan sulfate^{50,51} this suggests that higher expression of CD44 on the resistant variants could contribute to enhanced vaccinia infection, which is consistent with our findings. Furthermore, binding of CD44 to its cognate ligand hyaluronan initiates activation of several receptor tyrosine kinases (RTKs), non-RTKs [SRC (Src)], and cytoskeleton linker proteins [reviewed in ref⁶¹]. This complex cross-talk results in activation of PI3K-Akt and ERK that correlates with tumor progression and drug resistance^{51,52}, which are also known to augment vaccinia replication24,25. These latter findings are supported by at least 10-fold higher viral yields following infection of tumorigenic HeLa cells than those obtained following infection of embryo fibroblasts²⁵ and requirements of the MEK/ERK pathway for maximal vaccinia replication during productive infection in permissive cells, as both pharmacological and genetic inhibition of MEK/ERK resulted in decreases in viral yield^{24,53}. In addition, the observed downregulation of CD44 expression in ID8-R-infected cells suggests a direct interaction between CD44 and vaccinia (data not shown).

The present finding that a synergistic interaction of vaccinia with DOX occurred in both murine and human PTX- and CBDCA-resistant ovarian cancer cell lines implies that common pathways may mediate the effect. Our results differed from those of Siurala, et al., reporting DOX-mediated increases in adenoviral replication in human and hamster soft-tissue sarcoma cells²⁰. In our studies, DOX inhibited OVV-mediated killing when added before the virus. This could be related to the ability of anthracyclines to stimulate the rapid production of type IIFNs¹⁹, which in turn upregulates a large number of IFN-stimulated genes (ISGs)⁵⁴ with antiviral activities, including Myxovirus resistance (MX) genes (reviewed in ref.⁵⁵). Therefore, the current findings suggest that ordering of OVV/DOX combination treatments may be specific to the selected virus, the chemosensitivity profile, and/or phenotype of the targeted tumor cells. An exogenous supply of type IIFNs was also shown to restore the chemotherapeutic responses to DOX in Tlr.3^{-/-} but not Ifnar2^{-/-} sarcomas growing in mice¹⁹, which was associated with robust MX1 expression, consistent with improved chemotherapeutic responses to anthracyclines in patients with breast cancer who have poor prognosis¹⁹. Thus, the vaccinia-induced IFN-β in tumor cultures could explain the augmented responses to DOX characterized by higher expression of CRT and phagocytosis of tumor cell debris by DCs. The latter events are also necessary for complete DC activation and CD8⁺ T cell priming against tumor antigens⁵⁶⁻⁵⁸. Although the exposure of CRT on the cell surface of tumor cells is an important factor in determining immunogenicity of dying tumor cells^{59,60}, a still-unresolved issue surrounding tumor growth involves the role that the immune system plays in resisting or eradicating the formation and progression of tumors⁴³. During this process, cancer cells may paralyze infiltrating CTLs by secreting immunosuppressive factors⁶¹ or by more subtle mechanisms that operate through the recruitment of immunosuppressive elements, including MDSCs and Tregs. Without intending to be bound by any particular theory, it is considered that the finding that combined CXCR4 antagonist-expressing virus and PLD inhibited intratumoral recruitment of MDSCs and Tregs while inducing antitumor immunity and tumor-free survival supports the latter argument. It is noteworthy that the ID8-R variants have been generated from parental tumor cells, which were recovered from syngeneic recipients. Herein, many distinct mechanisms of tumor cell escape from the immune system could contribute to outgrowth of the tumor mass, which may then display an altered cell phenotype.⁶² Therefore, the ability of ID8-R tumor cells to generate spontaneous WT1-specific immune responses after treatment with OVV-CXCR4-A-Fc and PLD raises the possibility that the combination treatment with the armed oncolytic virotherapy and PLD rendered the tumor cells immunogenic by treatment-induced ICD while the suppressive elements in the tumor stroma have been compromised through our interventions. Alternatively, it is also conceivable that the in vivo selection process altered an immunogenic phenotype of the drug-resistant variants by changing expression levels of some tumor-associated antigens.

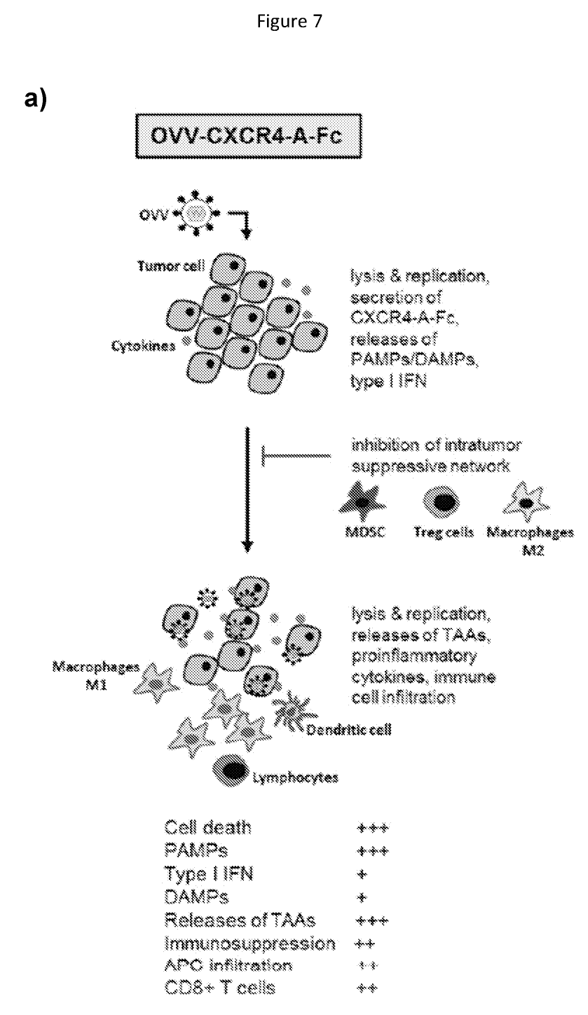

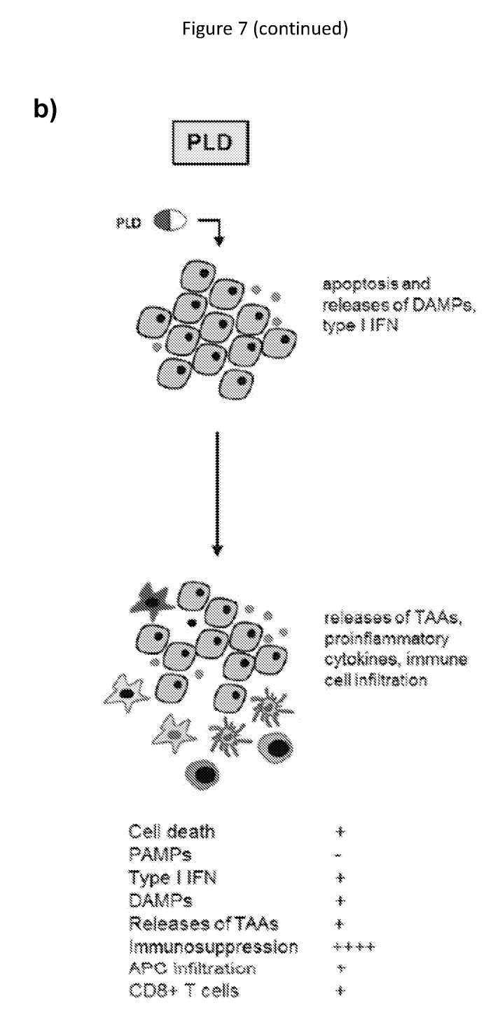

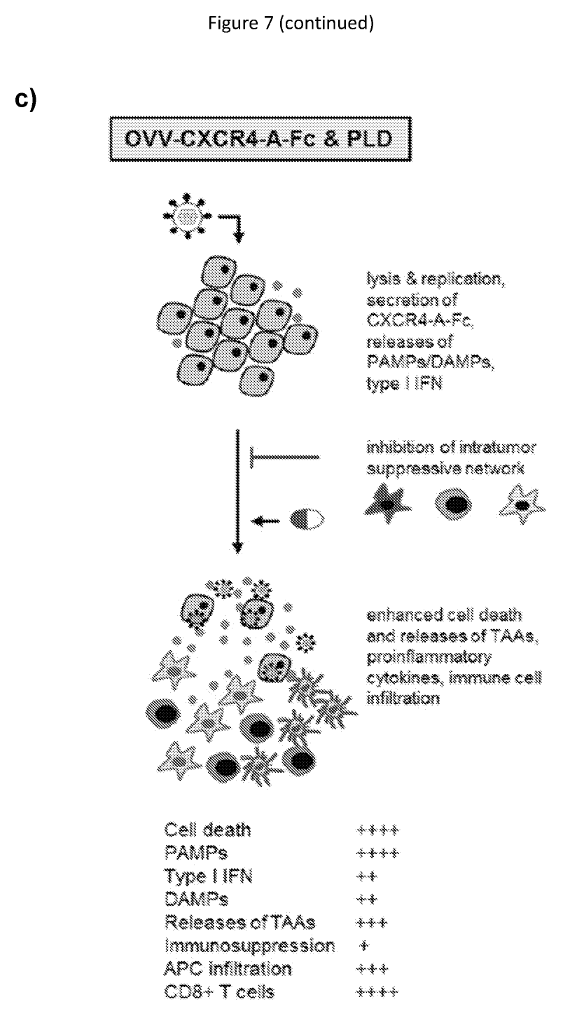

In conclusion, this disclosure demonstrates in non-limiting examples that showing vaccinia virus expressing the CXCR4 antagonist synergizes with DOX in killing PTX- and CBDCA-resistant variants of ovarian cancer and inhibits metastatic spread of the tumor by reducing tumor load and induction of antitumor immune responses as depicted in FIG. 7.

The following materials and methods were used to obtain the results discussed herein.

Animals and cell lines.

Female C57BL/6 and C.B-Igh-1b/IcrTac-Prkdc SCID mice, 6-8 wk of age, were obtained from Charles River (Wilmington, Mass.) and the Laboratory of Animal Resources at Roswell Park Cancer Institute (RPCI), Buffalo, N.Y., respectively. Experimental procedures were performed in compliance with protocols approved by the Institutional Animal Care and Use Committee of the RPCI. The parental ID8 mouse ovarian epithelial cells derived from spontaneous malignant transformation of C57BL/6 MOSE cells⁶³. The parental CAOV2 cell line was obtained from a collection maintained by the RPCI Department of Gynecologic Oncology. The genetic authenticity of CAOV2 cells was determined using microsatellite marker analysis⁶⁴ and the methylation status of the insulator protein CTCF within the insulin-like growth factor-II/H19 imprint center⁶⁵. The drug-resistant ID8-R and CAOV2-R variants were generated by isolating tumor cells from ascites of tumor-bearing syngeneic and SCID mice, respectively, which had been challenged with pFU-Luc2-Tomato lentiviral vector-transduced ID8-T and CAOV2 tumors³⁶ and treated daily with 35 μmol/kg of PTX delivered i.p. for a period of one week. Subsequently, the tumor variants were cultured in the presence of PTX (35.4 nM for ID8 and 118 nM for CAOV2) for three months until they gained a PTX-resistant phenotype. Interestingly, the PTX-resistant variants also acquired cross-resistance to CBDCA (2.6 Then, both cell lines were maintained in culture media supplemented with 59 nM PTX and 2.6 μM CBDCA, resulting in ID8-R and CAOV2-R variants. Human HuTK⁻ 143 fibroblasts, human cervical carcinoma HeLa cells, and African green monkey cell line CV-1 were obtained from the American Type Culture Collection (Manassas, Va.).

Viruses.

All vaccinia viruses used in this study are of the Western Reserve strain with disrupted thymidine kinase and vaccinia growth factor genes for enhanced cancer cell specificity. The generation and characterization of OVVs expressing the EGFP, Fc portion of murine IgG2a and the CXCR4 antagonist consisting of the eight amino acid corresponding to the N-terminal sequence of CXCL12 with modified P to G (KGVSLSYR) expressed in the context of murine (Fc) or human (hFc) fragment of IgG with disulfide bonds in a hinge region for preservation of a dimeric structure present in the CTCE-9908 template (KGVSLSYR-K-RYSLSVGK)³⁹ have been described^{29,36}.

Cytotoxicity assays.

Cells plated in 96-well plates were infected with serial dilutions of OVV-EGFP, OVV-Fc or treated with increasing doses of DOX (Sigma Aldrich, St. Louis, Mo.). For combination treatments, DOX was added at serial dilutions 12 h after viral infection, together with the virus, or 12 h before the infection. Cell survival was determined by 3-(4,5-Dimethylthiazol-2-yl)-2,5-diphenyltetrazolium bromide (MTT; Sigma) assays after 48 has described⁴⁵. Cell survival was calculated using the following formula: % cell survival=(absorbance value of treated cells/absorbance value of untreated control cells)×100%.

Viral replication.

Tumor cells seeded into 6-well plates were infected with OVV-EGFP at MOI=1 and incubated at 37° C. for 2 h. Then, the infection medium was removed and cells were incubated in fresh medium until cell harvest at 24, 48, and 72 h post-infection. In some experiments, DOX was added 12 h after viral infection. Viral particles from the infected cells were released by performing a quick freeze-thaw cycle and the titer was determined by plaque assays on CV-1 cell monolayers and recorded as PFU/million cells. EGFP expression in virally infected cultures was analyzed by flow cytometry or under fluorescent microscope (Zeiss Axiovert 40 CFL, 10×10) 24 h after the infection.

Tumorigenicity assays and immunogenicity of dying tumor cells.

Bulk cultures of parental and drug-resistant ID8 and CAOV2 tumor cells were injected i.p. using different numbers (5×10⁴-5×10⁶) into either syngeneic C57BL/6 or SCID mice (n=5-8) and monitored for tumor growth. To determine immunogenicity of dying tumor cells, 10⁶ treated ID8-R cells were inoculated subcutaneously (s.c.) in 100 μl of PBS into one flank of C57BL/6 six-week-old female mice, and 10⁶ untreated control cells were inoculated into the opposite flank 8 days later. Tumor growth was monitored by measuring s.c. tumors with a microcaliper and determining tumor volume (width×length×width/2=mm³).

Western blotting.

Cells were starved after reaching 80-90% of confluence when the medium was changed to 1% FBS and incubated for 12 h. Cells were solubilized in lysis buffer (Cell Signaling Technology, Danvers, Mass.) and samples of 25 μg of total protein, determined by Bradford assay, were separated on 4-20% Mini-Protean TGX gels (Bio-Rad Laboratories, Hercules, Calif.), transferred on to nitrocellulose membranes and incubated overnight with primary Abs against phospho-Akt (Ser473), phospho-Akt (Thr308), phospho-ERK1/2 (Thr202/Tyr204), Akt, ERK1/2 or GAPDH (Cell Signaling Technology). Bands were developed with HRP-labeled secondary Abs followed by Clarity Western ECL detection system (Bio-Rad Laboratories). Signal quantification was performed by densitometry analysis using a ChemiDoc MP imager and Image Lab software version 5.2.1 (Bio-Rad Laboratories).

The effect of DOX on expression of vaccinia virus antigens in infected cultures (MOI=10) was analyzed 24 h after the treatment. Cell lysates (25 μg/sample) were separated by SDS/PAGE (10% gel), transferred on to nitrocellulose membranes, and incubated for 2 h with OVV-specific mouse antiserum (1:2,000 dilution), prepared by immunizing C57BL/6 mice three time with 10⁸ PFU of OVV-Fc, or normal mouse serum as control. After washing, the membranes were incubated with HRP-labeled secondary antibody

Preparation of Media Collected from Virally-Infected Cells (CVN Media).

Tumor cells were infected with OVV-EGFP at MOI of 1, and media collected 24 h later were filtered and treated with UV light (365 nm for 3 min) in the presence of 10 μgml⁻¹ psolaren to inactivate the virus⁶⁶. A plaque assay was used to confirm lack of viral replication. Medium collected from uninfected cultures was used as a control. In some experiments, IFN-β levels in culture media were measured by ELISA (R&D Systems, Inc., Minneapolis, Minn.) according to the manufacturer's protocol.

Generation of BM-Derived DC and In Vitro Phagocytosis Assays.

BM cells were flushed from the tibias and femurs of C57BL/6 mice with culture medium composed of RPMI 1640 medium supplemented with 10% heat-inactivated fetal calf serum (Invitrogen, Carlsbad, Calif.), sodium pyruvate, 50 μM 2-mercaptoethanol (Sigma), 10 mM HEPES (pH 7.4), and penicillin/streptomycin (Invitrogen). After one centrifugation, BM cells were resuspended in Tris-ammonium chloride for 2 min to lyse red blood cells. After one more centrifugation, BM cells (1×10⁶ cells/ml) were cultured in medium supplemented with 10 ng/ml GM-CSF at 37° C. for 6 days. The medium was replenished every 2-3 days. After 7 days, the non-adherent and loosely adherent cells were harvested, washed and co-cultured with cell tracker-blue CMF₂HC (Thermo Fisher Scientific, Grand Island, N.Y.)-labeled tumor cells (1:1 ratio) for 12 h. At the end of the incubation, cells were harvested with versene, pooled with non-adherent cells present in the supernatant, washed and stained with CD11c-APC antibody. Phagocytosis was assessed by FACS analysis of double positive cells.

Treatment of established tumors.

C57BL/6 mice (n=6-10) were injected i.p. with 2×10⁵ ID8-R cells, whereas SCID mice (n=6) were injected i.p. with 2×10⁶ CAOV2-R cells. Treatment with OVV-CXCR4-A-Fc or OVV-Fc (10⁸ PFU delivered i.p.) was initiated 10 days later. In parallel experiments, tumor-bearing mice were treated with PLD alone (10 mg/kg, delivered i.v.) or PLD combined with OVV (delivered 8 days before, simultaneously of after virus injection). Tumor progression was monitored by bioluminescence imaging using the Xenogen IVIS Imaging System (PerkinElmer, Waltham, Mass.) after i.p. injection of 200 μl of Luciferin-D (150 mg/kg, Biosynth International Inc., Itasca, Ill.). For experiments in CAOV2-R-challenged SCID mice, animals were treated with lower titers of the virus (2.5×10⁷ PFU) and concentrations of PLD (5 mg/kg). Control mice received PBS or UV-inactivated virus. At the end of the experimental period corresponding to the development of bloody ascites in control mice, the tumor-bearing mice were sacrificed and organs were examined for tumor development and metastatic spread. Tumor and stromal cells were obtained from centrifuged cell pellets of ascites or peritoneal fluids collected from tumor-bearing mice after injection of 1 ml of PBS.

For adoptive transfer studies, C57BL/6 mice were injected s.c. with 10⁵ ID8-R tumor cells and treated 10 days later (tumor volume ˜100 mm³) by i.v. injection of 2×10⁷ splenocytes from tumor-bearing control mice or tumor-free mice with detectable WT1-specific immune responses after treatment with the OVV-CXCR4-A-Fc and PLD combination. Before the adoptive transfer, splenocytes were combined with LPS-matured WT1₁₂₆₋₁₃₄ peptide-pulsed BM-derived DCs (20:1) ratio as described⁴². Tumor growth was monitored by measuring s.c. tumors once to thrice a week with a microcaliper and determining tumor volume (width×length×width/2=mm³).

Flow cytometry.

Parental and drug-resistant ID8 tumor cells were analyzed by staining of single-cell suspensions with rat mAb against mouse CD44-PerCP-Cy5.5, whereas human CAOV2 tumor cells and their resistant variants were stained with mouse mAb against human CD44-PE (BD Pharmingen, San Jose, Calif.). The expression of CXCR4 on the surface of tumor cells was analyzed with rat mAb against mouse CXCR4-APC (BD Pharmingen) or human CXCR4-APC (eBioscience, San Diego, Calif.). The induction of apoptosis/necrosis in the resistant tumor cells treated with OVV-Fc (MOI=1), DOX (1 μM) alone or in combinations was assessed by staining with Annexin V-FITC and LIVE/DEAD fixable violet (Thermo Fisher Scientific) according to manufacturer's instruction. In some experiments, induction of apoptosis/necrosis was analyzed after incubating the resistant tumor cells for 24 h with the CXCR4-A-Fc fusion proteins (100 μg/ml) isolated from culture supernatant of infected cells by protein G column as described²⁹. Cultures incubated with the Fc portion of murine IgG2a served as control. Tumor cells were analyzed for cell surface expression of ecto-CRT by staining with rabbit anti-mouse CRT mAb (Abeam, Cambridge, Mass.) followed by staining with ΔPC-conjugated goat anti-rabbit secondary antibody (Santa Cruz Biotechnology, Santa Cruz, Calif.). The prevalence of SP cells in the parental and drug-resistant ID8 and CAOV2 cultures was determined on single-cell suspensions stained with Hoechst 33342 dye (Sigma) at a concentration of 5 μg/ml (37° C. for 2 h) as described²⁷. Cell analysis was performed on a LRS II flow cytometer (BD Biosciences, San Jose, Calif.). After excitation of the Hoechst dye at 350 nm and measurement of the fluorescence profile in dual-wavelength analysis (405/30 nm and 670/40 nm), the SP was defined as described⁶⁷.

The phenotypic analysis of G-MDSCs, Tregs, DCs, inflammatory monocytes/macrophages expressing IL-12 or IL-10, and CD8⁺ T lymphocytes were performed on single-cell suspensions prepared from peritoneal fluids collected 8 days after all treatments. The cells were stained with rat mAbs against mouse CD11b-APC, Ly6G-PE, Ly6C-FITC, CD45-APC-Cy7, CD4-PECy5, CD25-FITC, CD8-PECy5, IFN-γ-PE, CD11c-APC, CD86-FITC (BD Pharmingen), and Foxp3-AlexaFluor 647 (eBioscience, San Diego, Calif.), and F4/80-FITC (BioLegend, San Diego, Calif.). Percentages of CD8⁺ T cells expressing IFN-γ or CD4⁺ T cells expressing Foxp3 were determined by intracellular staining using BD Pharmingen™ Transcription Factor Buffer Set (BD Biosciences) according to the manufacturer's protocol. Percentages of CD11b/F4/80⁺macrophages expressing IL-12 or IL-10 were determined by intracellular staining with rat mAb against mouse IL-10-PE (BD Pharmingen) and anti-h/m IL-12/ILp35-PE Ab (R&D Systems).

To determine the percent of WT1₁₂₆₋₁₃₄/H-2D^{b} tetramer-specific CD8⁺ tumor-associated T lymphocytes, cells were stained with rat anti-mouse CD8-PECy5 mAb and a PE-labeled WT1₁₂₆₋₁₃₄/H-2D^{b} tetramer (MHC Tetramer Production Facility, Baylor College of Medicine, Houston, Tex.). Immune cells were gated on CD45⁺ viable cells for the analysis. For tetramer analysis, lymphocytes were also gated on cells that were negative for CD11b and Gr1 expression. Background staining was assessed using isotype control antibodies. Before specific antibody staining, cells were incubated with Fc blocker (anti-CD16/CD32 mAb) for 10 min followed by Live/Dead Fixable Violet Dead Cell stain kit (Thermo Fisher Scientific) to assess live/dead cells, and analyzed on a LRS II flow cytometer (BD Biosciences). Data analysis was performed using WinList 3D 7.1 (Verity Software House, Topsham, ME).

Statistical Analysis.

All statistical analyses were performed using GraphPad Prism 6 (GraphPad Software Inc., La Jolla, Calif.). Unless otherwise noted, data are presented as mean±S.D., combined with unpaired, two-tailed Student's t test. Kaplan-Meier survival plots were prepared and median survival times were determined for tumor-challenged groups of mice. Statistical differences in the survival across groups were assessed using the log-rank Mantel-Cox method. The threshold for statistical significance was set to P<0.05.

### REFERENCES

1 Bast, R C Jr, Hennessy, B and Mills, G B (2009). The biology of ovarian cancer: new opportunities for translation. Nat Rev Cancer 9: 415-428.
2 Jemal, A, Siegel, R, Ward, E, Hao, Y, Xu, J and Thun, M J (2009). Cancer statistics, 2009. Cancer J Clin 59: 225-249.
3 Thigpen, J T, Blessing, J A, Ball, H, Hummel, S J and Barrett, R. J (1994). Phase II trial of paclitaxel in patients with progressive ovarian carcinoma after platinum-based chemotherapy: a Gynecologic Oncology Group study. J Clin Oncol 12: 1748-1753.
4 Huang, E H, Heidt, D G, Li, C W and Simeone, D M (2007). Cancer stem cells: a new paradigm for understanding tumor progression and therapeutic resistance. Surgery 141: 415-419.
5 Alvero, A B, Chen, R, Fu, H H, Montagna, M, Schwartz, P E, Rutherford, T et al. (2009). Molecular phenotyping of human ovarian cancer stem cells unravels the mechanisms for repair and chemoresistance. Cell Cycle 8: 158-166.
6 Dean, M, Fojo, T and Bates, S (2005). Tumour stem cells and drug resistance. Nat Rev Cancer 5: 275-284.
7 Craveiro, V, Yang-Hartwich, Y, Holmberg, J C, Joo, W D, Sumi, N J, Pizzonia J et al. (2013). Phenotypic modifications in ovarian cancer stem cells following Paclitaxel treatment. Cancer Med 2: 751-762.
8 Sato, E, Olson, S H, Ahn, J, Bundy,B, Nishikawa, H, Qian, F et al. (2005). Intraepithelial CD8+ tumor-infiltrating lymphocytes and a high CD8+/regulatory T cell ratio are associated with favorable prognosis in ovarian cancer. Proc Natl Acad Sci USA 102: 18538-18543.
9 Odunsi, K, Qian, F, Matsuzaki, J, Mhawech-Fauceglia, P, Andrews, C, Hoffman, E W et al. (2007). Vaccination with an NY-ESO-1 peptide of HLA class I/II specificities induces integrated humoral and T cell responses in ovarian cancer. Proc Natl Acad Sci USA 104: 12837-12842.
10 Hwang, W T, Adams, S F, Tahirovic, E, Hagemann, I S and Coukos, G (2012). Prognostic significance of tumor-infiltrating T cells in ovarian cancer: a meta-analysis. Gynecol Oncol 124: 192-198.
11 Lichty, B D, Breitbach, C J, Stojdl, D F and Bell, J C (2014). Going viral with cancer immunotherapy. Nat Rev Cancer 14: 559-567.
12 Workenhe, S T, Simmons, G, Pol, J G, Lichty, B D, Halford, W P and Mossman, K L (2014). Immunogenic HSV-mediated oncolysis shapes the antitumor immune response and contributes to therapeutic efficacy. Mol Ther 22: 123-131.
13 Unterholzner, L, Keating, S E, Baran, M, Horan, K A, Jensen,S B, Sharma, S et al. (2010). IFI16 is an innate immune sensor for intracellular DNA. Nat Immunol 11: 997-1004.
14 Myskiw, C, Arsenio, J, Booy, E P, Hammett, C, Deschambault, Y, Gibson, S B et al. (2011). RNA species generated in vaccinia virus infected cells activate cell type-specific MDAS or RIG-I dependent interferon gene transcription and PKR dependent apoptosis. Virology 413: 183-193.
15 Duda, D G, Kozin, S V, Kirkpatrick, N D, Xu, L, Fukumura, D and Jain, R K (2011). CXCL12 (SDF1alpha)-CXCR4/CXCR7 pathway inhibition: an emerging sensitizer for anticancer therapies? Clin Cancer Res 17: 2074-2080.
16 Teicher, B A (2011). Antiangiogenic agents and targets: A perspective. Biochem Pharmacol 81: 6-12.
17 Pisano, C, Cecere, S C, Di Napoli, M, Cavaliere, C, Tambaro, R, Facchini, G et al. (2013). Clinical trials with pegylated liposomal Doxorubicin in the treatment of ovarian cancer. J Drug Deliv 2013: 898146.
18 Woo, S R, Corrales, L and Gajewski, T F (2015). The STING pathway and the T cell-inflamed tumor microenvironment. Trends Immunol 36: 250-256.
19 Sistigu, A, Yamazaki, T, Vacchelli, E, Chaba, K, Enot, D P, Adam, J et al. (2014). Cancer cell-autonomous contribution of type I interferon signaling to the efficacy of chemotherapy. Nat Med 20: 1301-1309.
20 Siurala, M, Bramante, S, Vassilev, L, Hirvinen, M, Parviainen, S, Tahtinen, S et al. (2015). Oncolytic adenovirus and doxorubicin-based chemotherapy results in synergistic antitumor activity against soft-tissue sarcoma. Int J Cancer 136: 945-954.
21 Zhang, S, Balch, C, Chan, M W, Lai, H C, Matei, D, Schilder, J M et al. (2008). Identification and characterization of ovarian cancer-initiating cells from primary human tumors. Cancer Res 68: 4311-4320.
22 Cioffi, M, D'Alterio, C, Camerlingo, R, Tirino, V, Consales, C, Riccio, A et al. (2015). Identification of a distinct population of CD133(+)CXCR4(+) cancer stem cells in ovarian cancer. Sci Rep 5: 10357.
23 Wang, G, Barrett, J W, Stanford, M, Werden, S J, Johnston, J B, Gao, X et al. (2006). Infection of human cancer cells with myxoma virus requires Akt activation via interaction with a viral ankyrin-repeat host range factor. Proc Natl Acad Sci USA 103: 4640-4645.
24 Andrade, A A, Silva, P N, Pereira, A C, De Sousa, L P, Ferreira, P C, Gazzinelli, R T et al. (2004). The vaccinia virus-stimulated mitogen-activated protein kinase (MAPK) pathway is required for virus multiplication. Biochem J 381: 437-446.
25 Soares, J A, Leite, F G, Andrade, L G, Torres, A A, De Sousa, L P, Barcelos, L S et al. (2009). Activation of the PI3K/Akt pathway early during vaccinia and cowpox virus infections is required for both host survival and viral replication. J Virol 83: 6883-6899.
26 Hirschmann-Jax, C, Foster, A E, Wulf, G G, Goodell, M A and Brenner, M K (2005). A distinct “side population” of cells in human tumor cells: implications for tumor biology and therapy. Cell Cycle 4: 203-205.
27 Hirschmann-Jax, C, Foster, A E, Wulf, G G, Nuchtern, J G, Jax, T W, Gobel, U et al. (2004). A distinct “side population” of cells with high drug efflux capacity in human tumor cells. Proc Natl Acad Sci USA 101: 14228-14233.
28 Stripecke, R, Carmen Villacres, M, Skelton, D, Satake, N, Halene, S and Kohn D (1999). Immune response to green fluorescent protein: implications for gene therapy. Gene Ther 6: 1305-1312.
29 Gil, M, Seshadri, M, Komorowski, M P, Abrams, S I and Kozbor, D (2013). Targeting CXCL12/CXCR4 signaling with oncolytic virotherapy disrupts tumor vasculature and inhibits breast cancer metastases. Proc Natl Acad Sci USA 110: E1291-1300.
30 McCart, J A, Ward, J M, Lee, J, Hu, Y, Alexander, H R, Libutti, S K et al. (2001). Systemic cancer therapy with a tumor-selective vaccinia virus mutant lacking thymidine kinase and vaccinia growth factor genes. Cancer Res 61: 8751-8757.
31 Kroemer, G, Galluzzi, L, Kepp, O and Zitvogel, L (2013). Immunogenic cell death in cancer therapy. Annu Rev Immunol 31: 51-72.
32 Gardai, S J, McPhillips, K A, Frasch, S C, Janssen, W J, Starefeldt, A, Murphy-Ullrich, J E et al. (2005). Cell-surface calreticulin initiates clearance of viable or apoptotic cells through trans-activation of LRP on the phagocyte. Cell 123: 321-334.
33 Ogden, C A, deCathelineau, A, Hoffmann, P R, Bratton, D, Ghebrehiwet, B, Fadok, V A et al. (2001). C1q and mannose binding lectin engagement of cell surface calreticulin and CD91 initiates macropinocytosis and uptake of apoptotic cells. J Exp Med 194: 781-795.
34 Obeid, M, Panaretakis, T, Joza, N, Tufi, R, Tesniere, A, van Endert, P et al. (2007). Calreticulin exposure is required for the immunogenicity of gamma-irradiation and UVC light-induced apoptosis. Cell Death Differ 14: 1848-1850.
35 Cojoc, M, Peitzsch, C, Trautmann, F, Polishchuk, L, Telegeev, G D and Dubrovska, A (2013). Emerging targets in cancer management: role of the CXCL12/CXCR4 axis. Onco Targets Ther 6: 1347-1361.
36 Gil, M, Komorowski, M P, Seshadri, M, Rokita, H, McGray, A J, Opyrchal, M et al. (2014). CXCL12/CXCR4 blockade by oncolytic virotherapy inhibits ovarian cancer growth by decreasing immunosuppression and targeting cancer-initiating cells. J Immunol 193: 5327-5337.
37 Wong, D and Korz, W (2008). Translating an Antagonist of Chemokine Receptor CXCR4: from bench to bedside. Clin Cancer Res 14: 7975-7980.
38 Hotte, S J, Hirte, H W, Moretto, P, Iacobucci, A, Wong, D, Korz, W at al. (2008). Final results of a Phase I/II study of CTCE-9908, a novel anticancer agent that inhibits CXCR4, in patients with advanced solid cancers. [abstract 405] EORTC/NCl/AACR Molecular Targets and Cancer Therapeutics 2008 conference; October 2008. Eur J Cancer 6: 127.
39 Huang, E H, Singh, B, Cristofanilli, M, Gelovani, J, Wei, C, Vincent, L et al. (2009). J Surg Res 155: 231-236.
40 Scotton, C J, Wilson, J L, Scott, K, Stamp, G, Wilbanks, G D, Fricker, S et al. (2002). Multiple actions of the chemokine CXCL12 on epithelial tumor cells in human ovarian cancer. Cancer Res 62: 5930-5938.
41 Hylander, B, Repasky, E, Shrikant, P, Intengan, M, Beck, A, Driscoll, D et al. (2006). Expression of Wilms tumor gene (WT1) in epithelial ovarian cancer. Gynecol Oncol 101: 12-17.
42 Gil, M, Bieniasz, M, Wierzbicki, A, Bambach, B J, Rokita, H and Kozbor, D (2009). Targeting a mimotope vaccine to activating Fcgamma receptors empowers dendritic cells to prime specific CD8+ T cell responses in tumor-bearing mice. J Immunol 183: 6808-6818.
43 Hanahan, D and Weinberg, R A (2011). Hallmarks of cancer: the next generation. Cell 144: 646-674.
44 Smith, L, Watson, M B, O'Kane, S L, Drew, P J, Lind, M J and Cawkwell, L (2006). The analysis of doxorubicin resistance in human breast cancer cells using antibody microarrays. Mol Cancer Ther 5: 2115-2120.
45 Wang, H, Chen, N G, Minev, B R and Szalay, A A (2012). Oncolytic vaccinia virus GLV-1h68 strain shows enhanced replication in human breast cancer stem-like cells in comparison to breast cancer cells. J Transl Med 10: 167-181.
46 Vanderplasschen, A, Mathew, E, Hollinshead, M, Sim, R B and Smith, G L (1998). Extracellular enveloped vaccinia virus is resistant to complement because of incorporation of host complement control proteins into its envelope. Proc Natl Acad Sci USA 95: 7544-7549.
47 Park, B H, Hwang, T, Liu, T C, Sze, D Y, Kim, J S, Kwon, H C et al. (2008). Use of a targeted oncolytic poxvirus, JX-594, in patients with refractory primary or metastatic liver cancer: a phase I trial. Lancet Oncol 9: 533-542.
48 Chung, C S, Hsiao, J C, Chang, Y S and Chang, W (1998). A27L protein mediates vaccinia virus interaction with cell surface heparan sulfate. J Virol 72: 1577-1585.
49 Hsiao, J C, Chung, C S and Chang, W (1998). Cell surface proteoglycans are necessary for A27L protein-mediated cell fusion: identification of the N-terminal region of A27L protein as the glycosaminoglycan-binding domain. J Virol 72: 8374-8379.
50 Greenfield, B, Wang, W C, Marquardt, H, Piepkorn, M, Wolff, E A, Aruffo, A et al. (1999). Characterization of the heparan sulfate and chondroitin sulfate assembly sites in CD44. J Biol Chem 274: 2511-2517.
51 Misra, S, Hascall, V C, Markwald, R R and Ghatak, S (2015). Interactions between Hyaluronan and Its Receptors (CD44, RHAMM) Regulate the Activities of Inflammation and Cancer. Front Immunol 6: 201-321.
52 Testa, J R and Bellacosa, A (2001). AKT plays a central role in tumorigenesis. Proc Natl Acad Sci USA 98: 10983-10985.
53 Silva, P N, Soares, J A, Brasil, B S, Nogueira, S V, Andrade, A A, de Magalhães, J C et al. (2006). Differential role played by the MEK/ERK/EGR-1 pathway in orthopoxviruses vaccinia and cowpox biology. Biochem J 398: 83-95.
54 Der, S D, Zhou, A, Williams, B R and Silverman, R H (1998). Identification of genes differentially regulated by interferon alpha, beta, or gamma using oligonucleotide arrays. Proc Natl Acad Sci USA 95: 15623-15628.
55 Melchjorsen, J (2013). Learning from the messengers: innate sensing of viruses and cytokine regulation of immunity—clues for treatments and vaccines. Viruses 5: 470-527.
56 Gajewski, T F (2015). The Next Hurdle in Cancer Immunotherapy: Overcoming the Non-T-Cell-Inflamed Tumor Microenvironment. Semin Oncol 42: 663-671.
57 Spranger, S, Bao, R and Gajewski, T F (2015). Melanoma-intrinsic beta-catenin signalling prevents anti-tumour immunity. Nature 523: 231-235.
58 Woo, S R, Corrales, L and Gajewski, T F (2015). Innate immune recognition of cancer. Annu Rev Immunol 33: 445-474.
59 Obeid, M, Tesniere, A, Panaretakis, T, Tufi, R, Joza, N, van Endert, P et al. (2007). Ecto-calreticulin in immunogenic chemotherapy. Immunol Rev 220: 22-34.
60 Obeid, M, Tesniere, A, Ghiringhelli, F, Fimia, G M, Apetoh, L, Perfettini, J L et al. (2007). Calreticulin exposure dictates the immunogenicity of cancer cell death. Nat Med 13: 54-61.
61 Yang, L, Pang, Y and Moses, H L (2010). TGF-beta and immune cells: an important regulatory axis in the tumor microenvironment and progression. Trends Immunol 31: 220-227.
62 Teng, M W, Swann, J B, Koebel, C M, Schreiber, R D and Smyth, M J (2008). Immune-mediated dormancy: an equilibrium with cancer. J Leukoc Biol 84: 988-993.
63 Janat-Amsbury, M M, Yockman, J W, Anderson, M L, Kieback, D G and Kim, S W (2006). Comparison of ID8 MOSE and VEGF-modified ID8 cell lines in an immunocompetent animal model for human ovarian cancer. Anticancer Res 26: 2785-2789.
64 Korch, C, Spillman, M A, Jackson, T A, Jacobsen, B M, Murphy, S K, Lessey, B A et al. (2012). DNA profiling analysis of endometrial and ovarian cell lines reveals misidentification, redundancy and contamination. Gynecol Oncol 127: 241-248.
65 Huang, Z and Murphy, S K (2013). Increased Intragenic IGF2 Methylation is Associated with Repression of Insulator Activity and Elevated Expression in Serous Ovarian Carcinoma. Front Oncol 3: 131-140.
66 Gil, M, Bieniasz, M, Seshadri, M, Fisher, D, Ciesielski, M J, Chen, Y et al. (2011). Photodynamic therapy augments the efficacy of oncolytic vaccinia virus against primary and metastatic tumours in mice. Br J Cancer 105: 1512-1521.
67 Goodell, M A, Brose, K, Paradis, G, Conner, A S and Mulligan R C (1996). Isolation and functional properties of murine hematopoietic stem cells that are replicating in vivo. J Exp Med 183: 1797-1806.

While the invention has been particularly shown and described with reference to specific embodiments (some of which are preferred embodiments), it should be understood by those having skill in the art that various changes in form and detail may be made therein without departing from the spirit and scope of the present invention as disclosed herein.

1. A method for inhibiting growth of tumor cells in an individual comprising administering to the individual a composition comprising a polynucleotide encoding a protein, wherein the protein encoded by the polynucleotide comprises an immunoglobulin Fc and an antagonist peptide of a receptor expressed by the tumor cells; and administering a chemotherapeutic agent to the individual, such that the growth of the tumor cells and/or metastasis of cancer cells is synergistically inhibited.

2. The method of claim 1, wherein the polynucleotide encoding the protein is present in a recombinant oncolytic vaccinia virus.

3. The method of claim 1, wherein the Fc is a human IgG1 Fc or human IgG3 Fc.

4. The method of claim 1, wherein the antagonist peptide comprises the sequence KGVSLSYR (SEQ ID NO:2).

5. The method of claim 1, wherein the antagonist peptide consists of the sequence KGVSLSYR (SEQ ID NO:2).

6. The method of claim 1, wherein the protein encoded by the polynucleotide comprises only one amino acid sequence of the antagonist peptide of the receptor expressed by the tumor cells.

7. The method of claim 6, wherein the only one amino acid sequence of the antagonist peptide of the receptor consists of the sequence KGVSLSYR (SEQ ID NO:2).

8. The method of claim 1, wherein the administration is a systemic administration.

9. The method of claim 1, wherein the polynucleotide is administered prior to the chemotherapeutic agent.

10. The method of claim 1, wherein the individual has a tumor that is resistant to the chemotherapeutic agent.

11. The method of claim 8, wherein the polynucleotide is administered prior to the chemotherapeutic agent.

12. The method of claim 8, wherein the individual has a tumor that is resistant to the chemotherapeutic agent.

13. The method of claim 9, wherein the administration is a systemic administration.

14. The method of claim 9, wherein the individual has a tumor that is resistant to the chemotherapeutic agent.

15. The method of claim 10, wherein the administration is a systemic administration.

16. The method of claim 10, wherein the polynucleotide is administered prior to the chemotherapeutic agent.

17. A method comprising sensitizing an individual to a cancer therapy comprising administering to the individual a composition comprising a polynucleotide encoding a protein, wherein the protein encoded by the polynucleotide comprises an immunoglobulin Fc and an antagonist peptide of a receptor expressed by the tumor cells; and subsequently administering the cancer therapy.

18. The method of claim 17, wherein the polynucleotide encoding the protein is present in a recombinant oncolytic vaccinia virus.

19. The method of claim 17, wherein the Fc is a human IgG1 Fc or human IgG3 Fc.

20. The method of claim 17, wherein the antagonist peptide comprises the sequence KGVSLSYR (SEQ ID NO:2).

21. The method of claim 17, wherein the antagonist peptide consists of the sequence KGVSLSYR (SEQ ID NO:2).

22. The method of claim 17, wherein the protein encoded by the polynucleotide comprises only one amino acid sequence of the antagonist peptide of the receptor expressed by the tumor cells.

23. The method of claim 17, wherein the only one amino acid sequence of the antagonist peptide of the receptor consists of the sequence KGVSLSYR (SEQ ID NO:2).

24. The method of claim 17, wherein the administration is a systemic administration.

25. The method of claim 17, wherein the cancer therapy comprises treatment with a chemotherapeutic agent and/or an adoptive immunotherapy.

26. The method of claim 17, wherein the administering the polynucleotide: i) inhibits formation of an intratumoral network, ii) improves immune cell infiltration of tumor, or a combination of i) and ii) occurs.


SEQUENCE LISTING (4 sequences)

SEQ ID NO: 1 - protein, 8 aa, human
KGVSLSYR

SEQ ID NO: 2 - protein, 17 aa, artificial sequence
  synthetic peptide
KGVSLSYRKRYSLSVGK

SEQ ID NO: 3 - protein, 320 aa, human
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYT

SEQ ID NO: 4 - protein, 377 aa, human
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK